SEQUENCE LISTING (4 sequences)

SEQ ID NO: 1 - protein, 127 aa, synthetic construct [sequence omitted]

SEQ ID NO: 2 - protein, 127 aa, synthetic construct
APARSPSPSTQPWEHVNAIQEALRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 3 - protein, 127 aa, synthetic construct [sequence omitted]

SEQ ID NO: 4 - protein, 126 aa, synthetic construct
APARSPSPSTQPWEHVNAIQEARRLLNLSRDTAAEMNETVEVISEMFDLQEPTCLQTRLELYKQGLRGSLTKLKGPLTMMASHYKQHCPPTPETSCATQTTFESFKENLKDFLLVIPFDCWEPVQE

(19) United States
(12) Patent Application Publication
(10) Pub. No.: US 2024/0361339 A1
(43) Pub. Date: Oct. 31, 2024

(54) BIOMARKERS FOR PARKINSON'S DISEASE

(71) Applicant: Board of Regents of The University of Nebraska, Lincoln, NE (US)

(72) Inventors: Howard GENDELMAN, Lincoln, NE (US); R. Lee MOSLEY, Lincoln, NE (US); Mai MOSTAFA, Lincoln, NE (US); Katherine OLSON, Lincoln, NE (US); Jatinkumar MACHHI, Lincoln, NE (US)

(21) Appl. No.: 18/688,019

(22) PCT Filed: Sep. 1, 2022
(86) PCT No.: PCT/US22/75837

§ 371 Date: Feb. 29, 2024

Related U.S. Application Data
(60) Provisional application No. 63/303,102, filed on Jan. 26, 2022.
(60) Provisional application No. 63/240,233, filed on Sep. 2, 2021.

(51) Int. Cl. G01N 33/68; A61K 38/19

(52) U.S. Cl. CPC G01N 33/6896; A61K 38/193; G01N 2333/4703; G01N 2333/70596; G01N 2333/90216; G01N 2333/9027; G01N 2333/912; G01N 2800/2835; G01N 2800/52

(57) ABSTRACT

The present disclosure relates to the treatment of Parkinson's disease with granulocyte-macrophage colony-stimulating factor, as well as diagnostic, prognostic and patient selection methods.

### CROSS REFERENCE TO RELATED APPLICATIONS

This application claims priority to U.S. Provisional Patent Application Nos. 63/240,233, filed Sep. 2, 2021 and 63/303,102, filed Jan. 26, 2022, the entire contents of each of which is hereby incorporated by reference for all purposes.

### GOVERNMENT SUPPORT

This invention was made with government support under Grant No. R01NS034239 awarded by the National Institutes of Health. The government has certain rights in the invention.

### FIELD

This disclosure relates to, in part, treatment and/or mitigation of Parkinson's disease, as well as diagnostic, prognostic and patient selection methods.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: Sequence_Listing_PNR-011PC/127114-5011.xml”; date recorded: Aug. 26, 2022; file size: 5,000 bytes).

### BACKGROUND

Neurodegenerative diseases are increasingly recognized as major causes of death and disability (including disability-adjusted life-years (DALYs; the sum of years of life lost [YLLs] and years lived with disability [YLDs]) worldwide. Globally, in 2016, neurological disorders were the leading cause of DALYs (˜276 million) and second leading cause of deaths (˜9.0 million). See Global Burden of Diseases, Injuries, and Risk Factors Study (GBD). Lancet Neurol 2019; 18:459-80.

Neurodegenerative disorders can be broadly classified by their clinical presentations, with extrapyramidal and pyramidal movement disorders and cognitive or behavioral disorders being the most common. Few patients have pure syndromes, with most having mixed clinical features. Although neurodegenerative diseases are typically defined by specific protein accumulations and anatomic vulnerability, neurodegenerative diseases share many fundamental processes associated with progressive neuronal dysfunction and death, such as proteotoxic stress and its attendant abnormalities in ubiquitin—proteasomal and autophagosomal/lysosomal systems, oxidative stress, programmed cell death, and neuroinflammation. See Dugger B N and Dickson D W. Cold Spring Harb Perspect Biol. 2017. 9 (7): a028035.

Parkinson's disease (PD) is a progressive neurodegenerative disorder characterized by a progressive loss of nigral dopaminergic neurons. The dominant presence of α-synuclein (α-syn) aggregates in intracellular inclusions, inside and outside the central nervous system (CNS), is a disease driver providing a signature for systemic disease with multiorgan involvement and consequent immune responses. Linkages between innate (monocytes and microglia) and adaptive (T cells) immunity, inflammation and disease are well established. The principal drivers are α-syn misfolding and aggregation, impairment of protein clearance, mitochondrial dysfunction, and inflammation. All of these affect dopaminergic neuronal function with secondary effects on noradrenergic, glutamatergic, serotonergic, and adenosine neuronal vitality. The disease is highlighted by the release of α-syn aggregates into the systemic circulation breaking the immune tolerance. In particular, activated monocytes enter the meninges and choroid plexus and provide early deleterious effects on neuroimmune homeostasis. Based on these findings, efforts to maintain immune homeostasis are attractive targets for PD-modifying therapies. One means to restore immune homeostasis, inside and outside the CNS, has focused on the immune transformation. Notably, a balanced transformation of monocytes-macrophages, effector T cells (Teff) to regulatory T cells (Treg), and the interactions between the two are of putative clinical benefit for PD as well as for Alzheimer's disease (AD), traumatic brain injury, stroke, and amyotrophic lateral sclerosis (ALS). The enhancement of Treg numbers and function leads to restoration of innate microglial homeostasis and control of neuroinflammation and neuroprotection. See Schabitz W R, et al. J Cereb Blood Flow Metab. 2008. 28, 29-43; Brochard V, et al. J Clin Invest. 2009. 119, 182-192; Jain S. Parkinsonism Relat Disord. 2011. 17, 77-83; Waschbisch, A, et al. J Immunol. 2016. 196, 1558-1567; Houser M C and Tansey M G. NPJ Parkinsons Dis. 2017. 3 (3); Rocha E M, et al. Neurobiol Dis. 2018. 109, 249-257; Harms A S, et al. Exp Neurol. 2018. 300, 179-187; Nissen S K et al. Mov Disord. 2019. 34, 1711-1721; Machhi J, et al. Mol Neurodegener. 2020. 15 (32); J. Jankovic J and Tan E K. J Neurol Neurosurg Psychiatry. 2020. 91, 795-808.

The increasing recognition that inflammation can play a critical role in neurodegenerative diseases of the CNS is highlighted by both adaptive versus innate immune responses being observed at various stages of neurodegenerative diseases. These differential immune responses may not only drive disease processes but could serve as therapeutic targets. Ongoing investigations into the specific inflammatory mechanisms that play roles in disease causation and progression have revealed lessons about inflammation-driven neurodegeneration understanding the advent of these diseases, as well as therapeutics to treat them. An increasing number of immunotherapeutic strategies that have been successful in MS are now being applied to other neurodegenerative diseases. Some approaches suppress CNS immune mechanisms, while others harness the immune system to clear deleterious products and cells. See Mamun A A and Liu F. Neurol Neurother. 2017. 2 (1); Chitnas T and Weiner H L. J Clin Invest. 2017. 127 (10): 3577-3587.

It is critical to note that not all immune responses in the CNS are detrimental, and in many cases, they actually aid repair and regeneration. For example, microglia clear debris after myelin damage and when this is impeded, delayed regeneration occurs. Immune activation is also crucial to limit neurotropic viral infections and removes necrotic cells following ischemia. Thus, microglia can exert dual roles in neurodegeneration, both as instigators of damage and as guardians of brain homeostasis. Besides microglia, T cells can also aid recovery during neurodegenerative diseases, although the exact mechanisms for this beneficial role of T cells are not clear. Detailed studies of neuroimmune interaction at both cellular and molecular levels have revealed complex interactions, demonstrating that immune cells can secrete both neurotoxic and neuroprotective molecules. As such, regulation of the immune response in neurological disorders has therapeutic value. See Neumann H et al. Brain. 2009. 132:288-95; Schwartz M et al. Neuroscience. 2009. 158:1133-42; Amor S et al. Immunology. 2010. 129 (2): 154-69.

The clinical and pathobiological diversity of Parkinson's disease has presented as a major challenge in the development of relevant biomarkers to monitor disease progression and disease-modifying therapies. Further, since therapeutic response can vary based on heterogeneous clinical and molecular phenotypes, a shift toward personalized or precision medicine approaches, including biomarker development and validation, has been thought to improve the management of many neurodegenerative diseases like PD.

Granulocyte Macrophage—Colony Stimulating Factor (GM-CSF) is a hematological growth factor that regulates the production, migration, proliferation, differentiation and function of hematopoietic cells. It was first identified as being able to induce, in vitro, the proliferation and differentiation of bone marrow progenitors into granulocytes and macrophages. In response to inflammatory stimuli, GM-CSF is released by various cell types including T lymphocytes, macrophages, fibroblasts and endothelial cells. GM-CSF then activates and enhances the production and survival of neutrophils, eosinophils, and macrophages. Native GM-CSF is usually produced near the site of action where it modulates in vitro proliferation, differentiation, and survival of hematopoietic progenitor cells, but is present in circulating blood in only picomolar concentrations (10⁻¹⁰ to 10⁻¹² M). Several studies have shown that GM-CSF has a wide range of functions across different tissues in its action on myeloid cells, and GM-CSF deletion/depletion approaches have indicated its potential as an important therapeutic target in several inflammatory and autoimmune disorders. See A Metcalf D. Immunol Cell Biology. 1987, 65:35-43; Gasson J C. Blood. 1991, 77:1131-1145; Shannon M F et al. Crit Rev Immunol. 1997, 17:301-323; Alexander W S. Int Rev Immunol. 1998, 16:651-682; Barreda D R et al. Dev Comp Immunol. 2004, 28:509-554; Lee K M C et al. Immunotargets Ther. 2020. 9:225-240.

Recombinant human granulocyte-macrophage colony-stimulating factor (rhu GM-CSF) has been approved by the FDA for the treatment of neutropenia, blood dyscrasias and malignancies like leukemia in combination with chemotherapies. In the clinic, GM-CSF used for treatment of neutropenia and aplastic anemia following chemotherapy greatly reduces the risk of infection associated with bone marrow transplantation. Its utility in myeloid leukemia treatment and as a vaccine adjuvant is also well established. See Dorr R T. Clin Therapeutics. 1993. 15 (1): 19-29; Armitage J O. Blood 1998, 92:4491-4508; Kovacic J C et al. J Mol Cell Cardiol. 2007, 42:19-33; Jacobs P P et al. Microbial Cell Factories 2010, 9:93.

The identification of specific biomarkers can be used for the diagnosis, prognosis, or theranosis of neurodegenerative diseases like Parkinson's disease. There remains a need for new and more effective biomarkers and combination treatments of Parkinson's disease.

### SUMMARY

Accordingly, in aspects, the present disclosure relates to a method for treating Parkinson's disease comprising: administering an effective amount of a composition comprising GM-CSF to a patient in need thereof, wherein the patient is characterized by a change in expression and/or activity of various biomarkers.

In aspects, the present disclosure relates to a method for treating one or more of Parkinson's Disease, Alzheimer's disease, traumatic brain injury, stroke, amyotrophic lateral sclerosis, traumatic brain injury, progressive supranuclear palsy, down syndrome cognition, and encephalitis, comprising: administering an effective amount of a composition comprising GM-CSF to a patient in need thereof, wherein the patient is characterized by a change in expression and/or activity of various biomarkers.

In aspects, the present disclosure relates to a method for treating one or more diseases or disorders characterized by an imbalance of balance between Teff and Treg, comprising: administering an effective amount of a composition comprising GM-CSF to a patient in need thereof, wherein the patient is characterized by a change in expression and/or activity of various biomarkers.

In aspects, there is provided a method of selecting a patient for treatment with an agent for Parkinson's disease and/or assessing the therapeutic response to an agent for Parkinson's disease, comprising determining the presence, absence or amount of one or more biomarkers in a biological sample from the patient, wherein the patient is suitable for the treatment if demonstrating a change in the expression and/or activity of the biomarkers relative to a pre-treated and/or undiseased state, and the agent comprises an effective amount of a granulocyte-macrophage colony-stimulating factor (GM-CSF).

In aspects, there is provided a method of selecting a patient for treatment with an agent for one or more of Parkinson's Disease, Alzheimer's disease, traumatic brain injury, stroke, amyotrophic lateral sclerosis, traumatic brain injury, progressive supranuclear palsy, down syndrome cognition, and encephalitis and/or assessing the therapeutic response to an agent for one or more of Parkinson's Disease, Alzheimer's disease, traumatic brain injury, stroke, amyotrophic lateral sclerosis, traumatic brain injury, progressive supranuclear palsy, down syndrome cognition, and encephalitis, comprising determining the presence, absence or amount of one or more biomarkers in a biological sample from the patient, wherein the patient is suitable for the treatment if demonstrating a change in the expression and/or activity of the biomarkers relative to a pre-treated and/or undiseased state, and the agent comprises an effective amount of a granulocyte-macrophage colony-stimulating factor (GM-CSF).

In aspects, there is provided a method of selecting a patient for treatment with an agent for one or more diseases or disorders characterized by an imbalance of balance between Teff and Treg and/or assessing the therapeutic response to an agent for one or more diseases or disorders characterized by an imbalance of balance between Teff and Treg, comprising determining the presence, absence or amount of one or more biomarkers in a biological sample from the patient, wherein the patient is suitable for the treatment if demonstrating a change in the expression and/or activity of the biomarkers relative to a pre-treated and/or undiseased state, and the agent comprises an effective amount of a granulocyte-macrophage colony-stimulating factor (GM-CSF).

In embodiments, the biomarker is selected from HMOX1, TLR2, TLR8, RELA (NF-kB p65), IKBGG, ATG3, ATG7, Leucine-rich repeat serine/threonine protein kinase 2 (LRRK2), GABARAPL2, RCOR1, GGA3, ALDH1A1, RFC1, BTF3L4, WBP2, EEA1, NCBP2, PEA15, MCM5, CLTA, VPS41, SRSF4, H2AFX, CD9, RFLNB, GLB1, KRT10, ACAA1, PCK2, ATP5F1D, ATP5PB, ATP5PF, ATP5PO, COX5B, COX7C, NDUFA2, NDUFB1, NDUFB4, NDUFS2, NDUFS3, NDUFS6, NDUFS7, SDHA, ATG3, ATG7, and GABARAPL2 optionally the biomarker is selected from selected from HMOX1, TLR2, TLR8, RELA, ATG7, LRRK2, and GABARAPL2.

In embodiments, the one or more of HMOX1, TLR2, TLR8, RELA, LRRK2, IKBGG, and ATP5F1D, ATP5PB, ATP5PF, ATP5PO, COX5B, COX7C, NDUFA2, NDUFB1, NDUFB4, NDUFS2, NDUFS3, NDUFS6, NDUFS7, and SDHA are downregulated during or after treatment with GM-CSF. In embodiments, one or more of HMOX1, TLR2, TLR8, RELA, and LRRK2 are downregulated during or after treatment with GM-CSF.

In embodiments, the one or more of HMOX1, TLR2, TLR8, RELA, LRRK2, IKBGG, and ATP5F1D, ATP5PB, ATP5PF, ATP5PO, COX5B, COX7C, NDUFA2, NDUFB1, NDUFB4, NDUFS2, NDUFS3, NDUFS6, NDUFS7, and SDHA are downregulated after one month, two months, three months, four months, five months and/or after six months of treatment with GM-CSF. In embodiments, the one or more of HMOX1, TLR2, TLR8, RELA, and LRRK2 are downregulated after one month, two months, three months, four months, five months and/or after six months of treatment with GM-CSF.

In embodiments, the one or more of ATG3, ATG7, and GABARAPL2 are upregulated during or after treatment with GM-CSF. In embodiments, the one or more of ATG7, and GABARAPL2 are upregulated during or after treatment with GM-CSF.

In embodiments, all of ATG3, ATG7, and GABARAPL2 are upregulated during or after treatment with GM-CSF. In embodiments, both of ATG7 and GABARAPL2 are upregulated during or after treatment with GM-CSF.

In embodiments, ATG3, ATG7, and GABARAPL2 are upregulated after one month, two months, three months, four months, five months and/or after six months of treatment with GM-CSF. In embodiments, ATG7 and GABARAPL2 are upregulated after one month, two months, three months, four months, five months and/or after six months of treatment with GM-CSF

In embodiments, (i) one or more of HMOX1, TLR2, TLR8, RELA, LRRK2, IKBGG, and ATP5F1D, ATP5PB, ATP5PF, ATP5PO, COX5B, COX7C, NDUFA2, NDUFB1, NDUFB4, NDUFS2, NDUFS3, NDUFS6, NDUFS7, and SDHA are downregulated during or after treatment with GM-CSF, optionally wherein one or more of HMOX1, TLR2, TLR8, RELA, and LRRK2 are downregulated during or after treatment with GM-CSF, and (ii) one or more of ATG3, ATG7, and GABARAPL2 are upregulated during or after treatment with GM-CSF, optionally wherein one or more of ATG7 and GABARAPL2 are upregulated during or after treatment with GM-CSF.

In embodiments, the biomarker is associated with one or more pathways selected from a neuroinflammation signaling pathway, IL-8 signaling pathway, production of nitric oxide and reactive oxygen species pathways, Integrin-Linked Kinase (ILK) signaling pathway, Sirtuin signaling pathway, and oxidative phosphorylation pathway.

In embodiments, the biomarker associated with a neuroinflammation signaling pathway, IL-8 signaling pathway, production of nitric oxide and reactive oxygen species pathways, Integrin-Linked Kinase (ILK) signaling pathway, and the oxidative phosphorylation pathway are downregulated or inhibited during or after treatment with GM-CSF.

In embodiments, the biomarker associated with a neuroinflammation signaling pathway, IL-8 signaling pathway, production of nitric oxide and reactive oxygen species pathways, Integrin-Linked Kinase (ILK) signaling pathway, and the oxidative phosphorylation pathway are downregulated or inhibited after about one month, about two months, about three months, about four months, about five months and/or after six months of treatment with GM-CSF.

In aspects, there is provided a method for treating Parkinson's disease in a patient, the method comprising the steps of identifying the patient having symptoms of Parkinson's disease; and determining the presence, absence or amount of one or more biomarkers in a biological sample from the patient; and administering an effective amount of a GM-CSF agent to the patient demonstrating a change in expression and/or activity of the one or more biomarkers relative to a pre-treated and/or undiseased state.

In embodiments, the biomarker is selected from HMOX1, TLR2, TLR8, RELA, IKBGG, ATG3, ATG7, LRRK2, GABARAPL2, RCOR1, GGA3, ALDH1A1, RFC1, BTF3L4, WBP2, EEA1, NCBP2, PEA15, MCM5, CLTA, VPS41, SRSF4, H2AFX, CD9, RFLNB, GLB1, KRT10, ACAA1, PCK2, ATP5F1D, ATP5PB, ATP5PF, ATP5PO, COX5B, COX7C, NDUFA2, NDUFB1, NDUFB4, NDUFS2, NDUFS3, NDUFS6, NDUFS7, SDHA, ATG3, ATG7, and GABARAPL2, optionally the biomarker is selected from HMOX1, TLR2, TLR8, RELA, ATG7, LRRK2, and GABARAPL2.

In embodiments, one or more of HMOX1, TLR2, TLR8, RELA, LRRK2, IKBGG, and ATP5F1D, ATP5PB, ATP5PF, ATP5PO, COX5B, COX7C, NDUFA2, NDUFB1, NDUFB4, NDUFS2, NDUFS3, NDUFS6, NDUFS7, and SDHA are downregulated during or after treatment with GM-CSF. In embodiments, HMOX1, TLR2, TLR8, RELA, and LRRK2 are downregulated during or after treatment with GM-CSF.

In embodiments, one or more of HMOX1, TLR2, TLR8, RELA, LRRK2, IKBGG, and ATP5F1D, ATP5PB, ATP5PF, ATP5PO, COX5B, COX7C, NDUFA2, NDUFB1, NDUFB4, NDUFS2, NDUFS3, NDUFS6, NDUFS7, and SDHA are downregulated after one month, two months, three months, four months, five months and/or after six months of treatment with GM-CSF. In embodiments, one or more of HMOX1, TLR2, TLR8, RELA, and LRRK2 are downregulated during or after treatment with GM-CSF are downregulated after one month, two months, three months, four months, five months and/or after six months of treatment with GM-CSF.

In embodiments, one or more of ATG3, ATG7, and GABARAPL2 are upregulated during or after treatment with GM-CSF. In embodiments, the one or more of ATG7, and GABARAPL2 are upregulated during or after treatment with GM-CSF. In embodiments, all of ATG3, ATG7, and GABARAPL2 are upregulated during or after treatment with GM-CSF. In embodiments, both of ATG7 and GABARAPL2 are upregulated during or after treatment with GM-CSF. In embodiments, one or more of ATG3, ATG7, and GABARAPL2 are upregulated after about one month, about two months, about three months, about four months, about five months and/or about six months of treatment with GM-CSF. In embodiments, one or more of ATG7 and GABARAPL2 are upregulated after about one month, about two months, about three months, about four months, about five months and/or about six months of treatment with GM-CSF.

In embodiments, (i) one or more of HMOX1, TLR2, TLR8, RELA, LRRK2, IKBGG, and ATP5F1D, ATP5PB, ATP5PF, ATP5PO, COX5B, COX7C, NDUFA2, NDUFB1, NDUFB4, NDUFS2, NDUFS3, NDUFS6, NDUFS7, and SDHA are downregulated during or after treatment with GM-CSF, optionally wherein one or more of HMOX1, TLR2, TLR8, RELA, and LRRK2 are downregulated during or after treatment with GM-CSF, and (ii) one or more of ATG3, ATG7, and GABARAPL2 are upregulated during or after treatment with GM-CSF, optionally wherein one or more of ATG7 and GABARAPL2 are upregulated during or after treatment with GM-CSF.

In embodiments, the biomarker is associated with one or more pathways selected from a neuroinflammation signaling pathway, IL-8 signaling pathway, production of nitric oxide and reactive oxygen species pathways, Integrin-Linked Kinase (ILK) signaling pathway, Sirtuin signaling pathway, and oxidative phosphorylation pathway.

In embodiments, the biomarker associated with a neuroinflammation signaling pathway, IL-8 signaling pathway, production of nitric oxide and reactive oxygen species pathways, Integrin-Linked Kinase (ILK) signaling pathway, and the oxidative phosphorylation pathway are downregulated or inhibited during or after treatment with GM-CSF.

In embodiments, the biomarker associated with a neuroinflammation signaling pathway, IL-8 signaling pathway, production of nitric oxide and reactive oxygen species pathways, Integrin-Linked Kinase (ILK) signaling pathway, and the oxidative phosphorylation pathway are downregulated or inhibited after one month, two months, three months, four months, five months and/or after six months of treatment with GM-CSF.

In aspects, there is provided a method for treating Parkinson's disease in a patient, the method comprising the steps of identifying the patient undergoing or having undergone treatment with a neurological agent for neurological symptoms and presenting as failed, intolerant, resistant, or refractory to the treatment with the neurological agent; and determining the presence, absence or amount of one or more of HMOX1, TLR2, TLR8, RELA, LRRK2, IKBGG, and ATP5F1D, ATP5PB, ATP5PF, ATP5PO, COX5B, COX7C, NDUFA2, NDUFB1, NDUFB4, NDUFS2, NDUFS3, NDUFS6, NDUFS7, and SDHA, ATG3, ATG7, and/or GABARAPL2; and administering an effective amount of a GM-CSF agent to the patient demonstrating an increased or high expression and/or activity of one or more of HMOX1, TLR2, TLR8, RELA, LRRK2, IKBGG, and ATP5F1D, ATP5PB, ATP5PF, ATP5PO, COX5B, COX7C, NDUFA2, NDUFB1, NDUFB4, NDUFS2, NDUFS3, NDUFS6, NDUFS7, and/or SDHA relative to a pre-treated and/or undiseased state; and/or demonstrating an decreased or low expression and/or activity of ATG3, ATG7, and/or GABARAPL2 relative to a pre-treated and/or undiseased state.

In aspects, there is provided a method for treating Parkinson's disease in a patient, the method comprising the steps of identifying the patient undergoing or having undergone treatment with a neurological agent for neurological symptoms and presenting as failed, intolerant, resistant, or refractory to the treatment with the neurological agent; and determining the presence, absence or amount of one or more of HMOX1, TLR2, TLR8, RELA, LRRK2, IKBGG, and ATP5F1D, ATP5PB, ATP5PF, ATP5PO, COX5B, COX7C, NDUFA2, NDUFB1, NDUFB4, NDUFS2, NDUFS3, NDUFS6, NDUFS7, and SDHA, ATG3, ATG7, and/or GABARAPL2; and administering an effective amount of a GM-CSF agent to the patient demonstrating a decreased or low expression and/or activity of one or more of HMOX1, TLR2, TLR8, RELA, LRRK2, IKBGG, and ATP5F1D, ATP5PB, ATP5PF, ATP5PO, COX5B, COX7C, NDUFA2, NDUFB1, NDUFB4, NDUFS2, NDUFS3, NDUFS6, NDUFS7, and/or SDHA relative to a pre-treated and/or undiseased state; and/or demonstrating an increased or high expression and/or activity of ATG3, ATG7, and/or GABARAPL2 relative to a pre-treated and/or undiseased state.

In aspects, there is provided a method for treating Parkinson's disease in a patient, the method comprising the steps of: identifying the patient undergoing or having undergone treatment with an neurological agent for neurological symptoms and presenting as failed, intolerant, resistant, or refractory to the treatment with the neurological agent; and determining an increase or decrease in expression and/or activity in biomarker from one or more pathways including the neuroinflammation signaling pathway, IL-8 signaling pathway, production of nitric oxide and reactive oxygen species pathways, Integrin-Linked Kinase (ILK) signaling pathway, Sirtuin signaling pathway, and the oxidative phosphorylation pathway; and administering an effective amount of a GM-CSF agent to the patient demonstrating an increased or high expression and/or activity in the neuroinflammation signaling pathway, IL-8 signaling pathway, production of nitric oxide and reactive oxygen species pathways, Integrin-Linked Kinase (ILK) signaling pathway, and the oxidative phosphorylation pathway relative to a pre-treated and/or undiseased state.

In aspects, there is provided a method for treating Parkinson's disease in a patient, the method comprising the steps of: (a) identifying the patient undergoing or having undergone treatment with an neurological agent for neurological symptoms and presenting as failed, intolerant, resistant, or refractory to the treatment with the neurological agent; (b) determining a decrease in expression and/or activity in biomarker from one or more pathways including the neuroinflammation signaling pathway, IL-8 signaling pathway, production of nitric oxide and reactive oxygen species pathways, Integrin-Linked Kinase (ILK) signaling pathway, and the oxidative phosphorylation pathway and/or an increase in expression and/or activity in biomarker from Sirtuin signaling pathway; and (c) administering an effective amount of a GM-CSF agent to the patient demonstrating a decreased or low expression and/or activity in the neuroinflammation signaling pathway, IL-8 signaling pathway, production of nitric oxide and reactive oxygen species pathways, Integrin-Linked Kinase (ILK) signaling pathway, and the oxidative phosphorylation pathway relative to a pre-treated and/or undiseased state.

In aspects, there is provided a method for treating Parkinson's disease in a patient, the method comprising the steps of: identifying the patient undergoing or having undergone treatment with an neurological agent for neurological symptoms and presenting as failed, intolerant, resistant, or refractory to the treatment with the neurological agent; and determining a decrease in expression and/or activity in biomarker from one or more pathways including the neuroinflammation signaling pathway, IL-8 signaling pathway, production of nitric oxide and reactive oxygen species pathways, Integrin-Linked Kinase (ILK) signaling pathway, and the oxidative phosphorylation pathway and/or an increase in expression and/or activity in biomarker from Sirtuin signaling pathway, and administering an effective amount of a GM-CSF agent to the patient demonstrating a decreased or low expression and/or activity in the neuroinflammation signaling pathway, IL-8 signaling pathway, production of nitric oxide and reactive oxygen species pathways, Integrin-Linked Kinase (ILK) signaling pathway, and the oxidative phosphorylation pathway relative to a pre-treated and/or undiseased state.

In aspects, there is provided a method for monitoring the regression, progression, disappearance or recurrence of symptoms of Parkinson's disease in a patient following treatment with a GM-CSF agent, the method comprising the steps of determining a baseline expression and/or activity level of one or more of the biomarkers at a first time point in a biological sample from the patient; determining the expression and/or activity level of one or more of the biomarkers at a second and subsequent time point in a biological sample; and determining if the expression and/or activity level of one or more of the biomarkers changes between the first and second time points, wherein the one or more biomarkers are selected from HMOX1, TLR2, TLR8, RELA, LRRK2, IKBGG, and ATP5F1D, ATP5PB, ATP5PF, ATP5PO, COX5B, COX7C, NDUFA2, NDUFB1, NDUFB4, NDUFS2, NDUFS3, NDUFS6, NDUFS7, and SDHA, ATG3, ATG7, and GABARAPL2.

In embodiments, the methods herein further comprising administering an effective amount of a drug or therapeutic to treat Parkinson's disease.

In embodiments, the patient is characterized by having one or more of oxidative stress, loss of neurite integrity, apoptosis, neuronal loss or/and inflammation response, cognitive impairment, cognitive decline, behavioral and personality changes, tremors, bradykinesia, rigidity, impaired posture and balance, loss of automatic movements, decrease in motor coordination, changes in speech, photophobia, difficulty controlling eye muscles, slowed saccadic eye movements, dysphagia, blepharospasm, fainting or lightheadedness due to orthostatic hypotension, dizziness, bladder control problems, well-formed visual hallucinations and delusions, changes in memory, concentration and judgement, memory loss, depression, irritability, anxiety, rapid eye movement (REM) sleep disorder, epileptic seizures, dysesthesia, numbness or tingling, spasticity, difficulty chewing or swallowing, muscle twitching and weakness in a limb, and/or prickling or tingling in feet or hands.

In embodiments, the presence, absence, or amount of the one or more biomarkers is determined by detection of protein and/or nucleic acids. In embodiments, the presence, absence, or amount of the one or more biomarkers is determined by one or more of ELISA, Luminex multiplex assay, immunohistochemical staining, western blotting, in-cell western, immunofluorescent staining, or fluorescent activating cell sorting (FACS). In embodiments, the presence, absence, or amount of the one or more biomarkers is determined by one or more of polymerase chain reaction (PCR) amplification reaction, reverse-transcriptase PCR analysis, quantitative real-time PCR, droplet-digital PCR (ddPCR), single-strand conformation polymorphism analysis (SSCP), mismatch cleavage detection, heteroduplex analysis, deoxyribonucleic (DNA) acid sequencing, ribonucleic acid (RNA) sequencing, Northern blot analysis, in situ hybridization, array analysis, and restriction fragment length polymorphism analysis. In embodiments, the presence, absence, or amount of the one or more biomarkers is determined by single-cell RNA sequencing and/or next generation sequencing (NGS) methods. In embodiments, the presence, absence, or amount of the one or more biomarkers is determined by RNA sequencing methods, e.g. single-cell RNA sequencing.

In embodiments, the biological sample is/or comprises blood, skin sample or tissue sample, plasma, serum, pus, urine, perspiration, tears, mucus, sputum, saliva, cerebrospinal fluid (CSF) and/or other body fluids. In embodiments, the biological sample is/or comprises monocytes. In embodiments, the biological sample is/or comprises monocyte population.

In embodiments, the method prevents, treats, and/or mitigates progression and/or development of Parkinson's disease in the patient. In embodiments, the method elicits a disease-modifying response and/or elicits temporarily or permanently slows down cognitive decline and/or causes an amelioration of neurodegenerative disease symptoms and/or slows the onset and/or development of the neurodegenerative disease or disorder and/or reverses or prevents chronic inflammation in the central nervous system (CNS) and/or decreases or mitigates the dysfunction of endogenous or exogenous CNS immune cells and/or decreases or mitigates the activation of CNS astrocytes and mononuclear phagocytes (e.g. perivascular macrophages and/or microglial cells), and/or decreases or mitigates or reverses astrogliopathy, and/or modulates or maintains or supports a glutamine-glutamate balance in the CNS, and/or decreases or mitigates or reverses chronic microglial cell activation, and/or decreases or reverses axonal damage, and/or decreases or prevents excessive production and/or signaling of one or more inflammatory cytokines and/or proteins, and/or decreases or prevents the formation of protein plaques, and/or causes a decrease or prevents taupathy.

In embodiments, the GM-CSF has an amino acid sequence of one of SEQ ID NO: 1, SEQ ID NO: 2, SEQ ID NO: 3, and SEQ ID NO: 4, or a variant of at least about 90%, or at least about 93%, or at least about 95%, or at least about 97%, or at least about 98% identity thereto. In embodiments, the GM-CSF is one of molgramostim, sargramostim, and regramostim. In embodiments, the GM-CSF is sargramostim. In embodiments, the GM-CSF is administered to via an intravenous route.

In embodiments, the method further comprises administering one or more additional therapeutic agents and/or neurological agents, selected from dopamine precursors such as levodopa, carbidopa (LODOSYN), dopamine agonists such as selegiline (ZELAPAR), MAO B inhibitors such as selegiline (ZELAPAR), catechol o-methyltransferase (COMT) inhibitors such as entacapone (COMTAN), anticholinergics such as benztropine (COGENTIN), amantadine, adenosine receptor antagonists (A2A receptor antagonists) such as istradefylline (NOURIANZ), and/or pimavanserin (NUPLAZID).

In aspects, there is provided a method for treating Parkinson's disease, comprising: selecting a patient having Parkinson's disease and one or more of changed expression and/or activity of one or more biomarkers relative to an undiseased state; and administering an effective amount of a composition comprising GM-CSF to the patient, wherein the one or more biomarkers are selected from HMOX1, TLR2, TLR8, RELA, LRRK2, IKBGG, and ATP5F1D, ATP5PB, ATP5PF, ATP5PO, COX5B, COX7C, NDUFA2, NDUFB1, NDUFB4, NDUFS2, NDUFS3, NDUFS6, NDUFS7, and SDHA, ATG3, ATG7, and GABARAPL2.

In embodiments, a changed expression and/or activity of the one or more biomarkers directs discontinued administration of GM-CSF. In embodiments, the levels of any of the biomarkers are assayed in a biological sample from the patient.

In embodiments, the biological sample comprises blood, tissue sample, plasma, serum, pus, urine, perspiration, tears, mucus, sputum, saliva, cerebrospinal fluid (CSF) and/or other body fluids.

In embodiments, the method prevents, treats, and/or mitigates progression and/or development of Parkinson's disease.

In embodiments, the method improves the symptoms of Parkinson's disease in the patient. In embodiments, the method causes a decrease in the sequelae of Parkinson's disease in the patient relative to before treatment.

In embodiments, the GM-CSF has an amino acid sequence of one of SEQ ID NO: 1, SEQ ID NO: 2, SEQ ID NO: 3, and SEQ ID NO: 4, or a variant of at least about 90%, or at least about 93%, or at least about 95%, or at least about 97%, or at least about 98% identity thereto. In embodiments, the GM-CSF is one of molgramostim, sargramostim, and regramostim. In embodiments, the GM-CSF is sargramostim. In embodiments, the GM-CSF is administered to via an intravenous route.

In embodiments, the method further comprises administering one or more additional therapeutic agents and/or neurological agents, selected from dopamine precursors such as levodopa, carbidopa (LODOSYN), dopamine agonists such as selegiline (ZELAPAR), MAO B inhibitors such as selegiline (ZELAPAR), catechol o-methyltransferase (COMT) inhibitors such as entacapone (COMTAN), anticholinergics such as benztropine (COGENTIN), amantadine, adenosine receptor antagonists (A2A receptor antagonists) such as istradefylline (NOURIANZ), and/or pimavanserin (NUPLAZID).

In aspects, there is provided a companion diagnostic, complementary diagnostic, or co-diagnostic test kit, comprising: an array of nucleic acids or proteins suitable for detection of one or more of HMOX1, TLR2, TLR8, RELA, LRRK2, IKBGG, and ATP5F1D, ATP5PB, ATP5PF, ATP5PO, COX5B, COX7C, NDUFA2, NDUFB1, NDUFB4, NDUFS2, NDUFS3, NDUFS6, NDUFS7, and SDHA, ATG3, ATG7, and GABARAPL2; and instructions for use.

In aspects, there is provided a companion diagnostic, complementary diagnostic, or co-diagnostic test kit, comprising reagents and instructions for use in one or more of methods described herein.

### BRIEF DESCRIPTION OF DRAWINGS

FIG. 1A illustrates pathway enrichment of differentially expressed proteins in monocytes of PD patients at 2 months after sargramostim treatment. Gene Ontology (GO)-term functional enrichment by 5 categories (immune response, biological process, cellular component, KEGG, and Reactome) was performed using Cytoscape in conjunction with the plug-in ClueGO.

FIG. 1B illustrates a canonical pathway enrichment analysis performed using IPA (Qiagen) showing differentially expressed proteins in monocytes in PD patients at 2 months after sargramostim treatment. Black arrow points to the state of canonical pathways illustrated in FIG. 1B; positive z-score (activation), negative z-score (inhibition), and light grey color (no activity pattern).

FIG. 2A illustrates pathway enrichment of differentially expressed proteins/genes in monocytes of PD patients at 6 months after sargramostim treatment. Gene Ontology (GO)-term functional enrichment by 5 categories (immune response, biological process, cellular component, KEGG, and Reactome) was performed using Cytoscape in conjunction with the plug-in ClueGO.

FIG. 2B illustrates a canonical pathway enrichment analysis performed using IPA (Qiagen) showing differentially expressed proteins in monocytes in PD patients at 6 months after sargramostim treatment. Black arrow points to the state of canonical pathways illustrated in FIG. 2B; positive z-score (activation), negative z-score (inhibition), and light grey color (no activity pattern).

FIG. 3. illustrates graphs depicting gene and protein expression of potential biomarkers in monocytes at 2 and 6 months after sargramostim treatment. The ddPCR assay was performed to determine the gene expression of LRRK2, HMOX1, TLR2, TLR8, RELA, ATG7, and GABARAPL2 at 2 (A) and 6 (B) months after starting the sargramostim treatment compared to baseline. Gene expression was normalized to HPRT1 and the ddPCR assay was performed 4 times (n=4 technical replicates). Western blot analysis was performed to determine the protein expression of β-actin, LRRK2, HMOX1, TLR2, TLR8, RELA, ATG7, and GABARAPL2 at 2 (C) and 6 (D) months after starting the sargramostim treatment compared to baseline. Protein expression was normalized to β-actin and densitometric quantification is shown. Western blot analysis was done thrice (n=3 technical replicates). Data represent mean±SD. Horizontal line in each image represents baseline expression; values above the line indicate upregulation while values below the line indicate downregulation.

FIG. 4A depicts the integration of scRNA-seq and proteomic data showing overlapping genes between scRNA-seq and proteomic data sets for patients 2003, 2004, and 2005 at 6 months after sargramostim treatment compared to baseline.

FIG. 4B shows a graph depicting the correlation of overlapped genes in both scRNA-seq and proteomic data sets for patients 2003, 2004, and 2005 at 6 months after sargramostim treatment compared to baseline. Correlation was determined using Pearson product-moment correlation coefficient (r).

FIG. 5A shows graphs depicting the correlation between gene expression of LRRK2, HMOX1, TLR2, TLR8, RELA, ATG7, and GABARAPL2 and change in MDS-UPDRS III score. r=Pearson product-moment correlation coefficient.

FIG. 5B shows graphs depicting the correlation between gene expression of LRRK2, HMOX1, TLR2, TLR8, RELA, ATG7, and GABARAPL2 and raw MDS-UPDRS III score. r=Pearson product-moment correlation coefficient.

FIG. 5C shows a multiple linear regression analysis of effect of gene expression of LRRK2, HMOX1, TLR2, TLR8, and ATG7 on change in MDS-UPDRS III score.

FIG. 5D shows a multiple linear regression analysis of effect of gene expression of LRRK2, HMOX1, TLR2, TLR8, and ATG7 on raw MDS-UPDRS III score. r=regression coefficient.

FIG. 6A shows graphs depicting the correlation between protein expression of LRRK2, RELA, and ATG7 and change in MDS-UPDRS III score. r=Pearson product-moment correlation coefficient.

FIG. 6B shows graphs depicting the correlation between protein expression of LRRK2, RELA, and ATG7 and raw MDS-UPDRS III score. r=Pearson product-moment correlation coefficient.

FIG. 6C shows a multiple linear regression analysis of effect of protein expression of LRRK2, HMOX1, RELA, and GABARAPL2 on change in MDS-UPDRS III score. r=regression coefficient.

FIG. 6D shows a multiple linear regression analysis of effect of protein expression of TLR2, TLR8, and ATG7 on raw MDS-UPDRS III score. r=regression coefficient.

### DETAILED DESCRIPTION

The present disclosure relates to, in part, to the use of GM-CSF as an effective treatment for Parkinson's disease, selected using specific biomarkers as a predictive clinical markers of disease sequelae and responsiveness to current therapy, e.g. with or without an agent to treat Parkinson's disease.

In aspects, the present disclosure relates to an improved method of selecting a patient in need of therapy for one or more of Parkinson's Disease, Alzheimer's disease, traumatic brain injury, stroke, amyotrophic lateral sclerosis, traumatic brain injury, progressive supranuclear palsy, down syndrome cognition, encephalitis, and/or one or more of indications characterized by an imbalance of balance between Teff and Treg and/or assessing the therapeutic response to an agent for one or more of Parkinson's Disease, Alzheimer's disease, traumatic brain injury, stroke, amyotrophic lateral sclerosis, traumatic brain injury, progressive supranuclear palsy, down syndrome cognition, encephalitis, and/or one or more of indications characterized by an imbalance of balance between Teff and Treg. In one aspect, the present disclosure relates to improved treatments for one or more of Parkinson's Disease, Alzheimer's disease, traumatic brain injury, stroke, amyotrophic lateral sclerosis, traumatic brain injury, progressive supranuclear palsy, down syndrome cognition, encephalitis, and/or one or more of indications characterized by an imbalance of balance between Teff and Treg in a patient based on select predictive biomarkers. For instance, in embodiments, evaluation of the presence, absence, levels or activity of one or more biomarkers such as HMOX1, TLR2, TLR8, RELA, IKBGG, ATG3, ATG7, LRRK2, GABARAPL2, RCOR1, GGA3, ALDH1A1, RFC1, BTF3L4, WBP2, EEA1, NCBP2, PEA15, MCM5, CLTA, VPS41, SRSF4, H2AFX, CD9, RFLNB, GLB1, KRT10, ACAA1, PCK2, ATP5F1D, ATP5PB, ATP5PF, ATP5PO, COX5B, COX7C, NDUFA2, NDUFB1, NDUFB4, NDUFS2, NDUFS3, NDUFS6, NDUFS7, SDHA, ATG3, ATG7, and/or GABARAPL2, optionally one or more biomarkers such as HMOX1, TLR2, TLR8, RELA, ATG7, LRRK2, and GABARAPL2, informs or predicts the disease state in the patient and, without limitation, directs the administration of GM-CSF to a patient with one or more of Parkinson's Disease, Alzheimer's disease, traumatic brain injury, stroke, amyotrophic lateral sclerosis, traumatic brain injury, progressive supranuclear palsy, down syndrome cognition, encephalitis, and/or one or more of indications characterized by an imbalance of balance between Teff and Treg. In other embodiments, evaluation of biomarkers from one or more pathways including the neuroinflammation signaling pathway, IL-8 signaling pathway, nitric oxide and reactive oxygen species pathways, Integrin-Linked Kinase (ILK) signaling pathway, Sirtuin signaling pathway, and the oxidative phosphorylation pathway informs or predicts the disease state in the patient and, without limitation, directs the administration of GM-CSF to a patient with one or more of Parkinson's Disease, Alzheimer's disease, traumatic brain injury, stroke, amyotrophic lateral sclerosis, traumatic brain injury, progressive supranuclear palsy, down syndrome cognition, encephalitis, and/or one or more of indications characterized by an imbalance of balance between Teff and Treg.

In aspects, the biomarkers relates to one or more proteins and/or biomarkers and signaling and/or regulatory pathways. In aspects, the biomarkers of the present disclosure is also used in combination with other one or more of Parkinson's Disease, Alzheimer's disease, traumatic brain injury, stroke, amyotrophic lateral sclerosis, traumatic brain injury, progressive supranuclear palsy, down syndrome cognition, encephalitis, and/or one or more of indications characterized by an imbalance of balance between Teff and Treg biomarkers to help monitor disease progression and response to therapy.

Accordingly, in aspects, the disclosure provides methods for treating one or more of Parkinson's Disease, Alzheimer's disease, traumatic brain injury, stroke, amyotrophic lateral sclerosis, traumatic brain injury, progressive supranuclear palsy, down syndrome cognition, encephalitis, and/or one or more of indications characterized by an imbalance of balance between Teff and Treg by evaluating select clinical biomarkers.

### Compositions

In embodiments, the present disclosure pertains to pharmaceutical compositions comprising the compositions, e.g. GM-CSF and/or an additional therapeutics to treat a Parkinson's disease.

In embodiments, the additional neurological agent and/or additional therapeutic agent is selected from selected from dopamine precursors such as levodopa, carbidopa (LODOSYN), dopamine agonists such as selegiline (ZELAPAR), MAO B inhibitors such as selegiline (ZELAPAR), catechol o-methyltransferase (COMT) inhibitors such as entacapone (COMTAN), anticholinergics such as benztropine (COGENTIN), amantadine, adenosine receptor antagonists (A2A receptor antagonists) such as istradefylline (NOURIANZ), and/or pimavanserin (NUPLAZID).

In embodiments, the neurological agent and/or additional therapeutic agent to treat Parkinson's disease is an antibody or antibody format which is selected from one or more of a monoclonal antibody, polyclonal antibody, antibody fragment, Fab, Fab′, Fab′-SH, F(ab′)2, Fv, single chain Fv, diabody, linear antibody, bispecific antibody, multispecific antibody, chimeric antibody, humanized antibody, human antibody, and fusion protein comprising the antigen-binding portion of an antibody.

### Compositions of GM-CSF

GM-CSF, in embodiments, includes any pharmaceutically safe and effective GM-CSF, or any derivative thereof having the biological activity of GM-CSF. In embodiments, the GM-CSF is rhu GM-CSF, such as sargramostim (LEUKINE). Sargramostim is a biosynthetic, yeast-derived, recombinant human GM-CSF, having a single 127 amino acid glycoprotein that differs from endogenous human GM-CSF by having a leucine instead of a proline at position 23. Other natural and synthetic GM-CSFs, and derivatives thereof having the biological activity of natural human GM-CSF, may be equally useful in embodiments.

In embodiments, the GM-CSF is produced or producible in bacteria, yeasts, plants, insect cells, and mammalian cells. In embodiments, the GM-CSF is produced or producible in Escherichia coli cells. In embodiments, the GM-CSF is produced or producible in yeast cells. In embodiments, the GM-CSF is produced or producible in Chinese hamster ovary cells (CHO). In embodiments, the GM-CSF is not produced in E. coli cells. In embodiments, the GM-CSF is produced in a cell that allows for glycosylation, e.g. yeast or CHO cells.

In embodiments, the GM-CSF has an amino acid sequence of SEQ ID NO: 1, or a variant of at least about 90%, or at least about 93%, or at least about 95%, or at least about 97%, or at least about 98% identity thereto. In embodiments, the GM-CSF has an amino acid sequence of SEQ ID NO: 2, SEQ ID NO: 3, and SEQ ID NO: 4, or a variant of at least about 90%, or at least about 93%, or at least about 95%, or at least about 97%, or at least about 98% identity thereto. In embodiments, the GM-CSF is one of, sargramostim, molgramostim, and regramostim. In embodiments, the GM-CSF is sargramostim.

Without wishing to be bound by theory, the core of hGM-CSF consists of four helices that pack at angles. Crystal structures and mutagenic analysis of rhGM-CSF (Rozwarski D A et al., Proteins 26:304-13, 1996) showed that, in addition to apolar side chains in the protein core, 10 buried hydrogen bonding residues involve intramolecular hydrogen bonding to main chain atoms that were better conserved than residues hydrogen bonding to other side chain atoms; 24 solvation sites were observed at equivalent positions in the two molecules in the asymmetric unit, and the strongest among these was located in clefts between secondary structural elements. Two surface clusters of hydrophobic side chains are located near the expected receptor binding regions. Mutagenesis of residues on the helix A/helix C face confirmed the importance of certain Glu, Gly, and Gln residues. These residues are therefore not to be substituted in the functional substitution variants of hGM-CSF for use in the present disclosure and these helices are to be retained in a functional fragments or deletion variants of hGM-CSF for use in this disclosure. Further, in embodiments, one of ordinary skill can reference UniProtKB entry P04141 for structure information to inform the identity of variants.

The N-terminal helix of hGM-CSF governs high affinity binding to its receptor (Shanafelt A B et al., EMBO J 10:4105-12, 1991) Transduction of the biological effects of GM-CSF requires interaction with at least two cell surface receptor components, (one of which is shared with the cytokine IL-5). The above study identified receptor binding determinants in GM-CSF by locating unique receptor binding domains on a series of human-mouse hybrid GM-CSF cytokines. The interaction of GM-CSF with the shared subunit of their high affinity receptor complexes was governed by a very small part of the peptide chains. The presence of a few key residues in the N-terminal α-helix of was sufficient to confer specificity to the interaction.

In embodiments, the amino acid mutations are amino acid substitutions, and may include conservative and/or non-conservative substitutions.

“Conservative substitutions” may be made, for instance, based on similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic: Met, Ala, Val, Leu, Ile; (2) neutral hydrophilic: Cys, Ser, Thr; Asn, Gln; (3) acidic: Asp, Glu; (4) basic: His, Lys, Arg; (5) residues that influence chain orientation: Gly, Pro; and (6) aromatic: Trp, Tyr, Phe.

As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices.

As used herein, “non-conservative substitutions” are defined as exchanges of an amino acid by another amino acid listed in a different group of the six standard amino acid groups (1) to (6) shown above.

In embodiments, the substitutions may also include non-classical amino acids (e.g. selenocysteine, pyrrolysine, N-formylmethionine β-alanine, GABA and δ-Aminolevulinic acid, 4-aminobenzoic acid (PABA), D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic t-butylglycine, acid, t-butylalanine, phenylglycine, cyclohexylalanine, β-alanine, fluoro-amino acids, designer amino acids such as β methyl amino acids, C α-methyl amino acids, N α-methyl amino acids, and amino acid analogs in general).

Modification of the amino acid sequences may be achieved using any known technique in the art e.g., site-directed mutagenesis or PCR based mutagenesis. Such techniques are described, for example, in Sambrook et al., Molecular Cloning: A Laboratory Manual, Cold Spring Harbor Press, Plainview, N.Y., 1989 and Ausubel et al., Current Protocols in Molecular Biology, John Wiley & Sons, New York, N.Y., 1989. Without wishing to be bound by theory, the degree of glycosylation of biosynthetic GM-CSFs appears to influence half-life, distribution, and elimination. (Lieschke and Burgess, N. Engl. J. Med. 327:28-35, 1992; Dorr, R. T., Clin. Ther. 15:19-29, 1993; Horgaard et al., Eur. J. Hematol. 50:32-36, 1993). In embodiments, the present GM-CSF molecules are glycosylated.

### Biomarker

In aspects, the present methods relate to the utility of novel predictive biomarkers to determine the use of GM-CSF in the treatment of Parkinson's disease.

In aspects, the present methods relate to the utility of novel predictive biomarkers to determine the use of GM-CSF in the treatment of one or more of Parkinson's Disease, Alzheimer's disease, traumatic brain injury, stroke, amyotrophic lateral sclerosis, traumatic brain injury, progressive supranuclear palsy, down syndrome cognition, and encephalitis.

In aspects, the present methods relate to the utility of novel predictive biomarkers to determine the use of GM-CSF in the treatment of one or more diseases or disorders characterized by an imbalance of balance between Teff and Treg.

In one aspect, the present disclosure relates to an improved method of selecting a patient in need of therapy for Parkinson's disease. In another aspect, the present disclosure relates to improved treatments for Parkinson's disease in a patient based on select predictive biomarkers. In other aspects, an assessment of the patient having failed or being intolerant or refractory to a treatment for Parkinson's disease comprises measuring of a biomarker in a biological sample of the patient.

In aspects, there is provided a method of selecting a patient for treatment with an agent for one or more of Parkinson's Disease, Alzheimer's disease, traumatic brain injury, stroke, amyotrophic lateral sclerosis, traumatic brain injury, progressive supranuclear palsy, down syndrome cognition, and encephalitis and/or assessing the therapeutic response to an agent for one or more of Parkinson's Disease, Alzheimer's disease, traumatic brain injury, stroke, amyotrophic lateral sclerosis, traumatic brain injury, progressive supranuclear palsy, down syndrome cognition, and encephalitis, comprising determining the presence, absence or amount of one or more biomarkers in a biological sample from the patient, wherein the patient is suitable for the treatment if demonstrating a change in the expression and/or activity of the biomarkers relative to a pre-treated and/or undiseased state, and the agent comprises an effective amount of a granulocyte-macrophage colony-stimulating factor (GM-CSF).

In aspects, there is provided a method of selecting a patient for treatment with an agent for one or more diseases or disorders characterized by an imbalance of balance between Teff and Treg and/or assessing the therapeutic response to an agent for one or more diseases or disorders characterized by an imbalance of balance between Teff and Treg, comprising determining the presence, absence or amount of one or more biomarkers in a biological sample from the patient, wherein the patient is suitable for the treatment if demonstrating a change in the expression and/or activity of the biomarkers relative to a pre-treated and/or undiseased state, and the agent comprises an effective amount of a granulocyte-macrophage colony-stimulating factor (GM-CSF).

In embodiments, an assessment of the patient with Parkinson's disease comprises measuring a variety of patient parameters. In embodiments, the patient biological sample may be analyzed using, e.g. immunohistochemical or immunofluorescence techniques may be used to evaluate the immune infiltrate, for example, immune subsets such as, CD4⁺ Th cells (T helper cells), IL-17-producing CD4⁺ Th cells (Th17 cells), CD8⁺ T cells (cytotoxic T cells), and systemic or circulating intermediate monocytes. In embodiments, polychromatic flow cytometry can be used to measure multiple surface and intracellular markers, allowing characterization of cell phenotype and activation state. In embodiments, whole blood can be used to evaluate changes in cell count with therapy or changes in cytokine levels, for example IL-1, IL-4, IL-6, IL-10, IL-12, IL-18, IL-33, IFN-g, IP-10, M-CSF, TGF-b, VEGF, and TNFα. In embodiments, deep sequencing techniques can be used to yield quantification of changes in individual cell clonotypes.

In embodiments, an assessment of the patient with Parkinson's disease comprises measuring the presence, absence, or amount of various biomarkers in a biological sample of the patient.

In embodiments, the present disclosure relates to a method for treating Parkinson's disease in a patient, wherein one or more of, e.g. about 1, or about 2, or about 3, or about 4, or about 5, or about 10, or about 15, or about 20, or about 25, or about 30, or about 35, or about 40, or about 45, of HMOX1, TLR2, TLR8, RELA, IKBGG, ATG3, ATG7, LRRK2, GABARAPL2, RCOR1, GGA3, ALDH1A1, RFC1, BTF3L4, WBP2, EEA1, NCBP2, PEA15, MCM5, CLTA, VPS41, SRSF4, H2AFX, CD9, RFLNB, GLB1, KRT10, ACAA1, PCK2, ATP5F1D, ATP5PB, ATP5PF, ATP5PO, COX5B, COX7C, NDUFA2, NDUFB1, NDUFB4, NDUFS2, NDUFS3, NDUFS6, NDUFS7, SDHA, ATG3, ATG7, and/or GABARAPL2. In embodiments, the present disclosure relates to a method for treating Parkinson's disease in a patient, wherein one or more of, e.g. about 1, or about 2, or about 3, or about 4, or about 5 of HMOX1, TLR2, TLR8, RELA, ATG7, LRRK2, and GABARAPL2, are used as a biomarker for predicting or determining the need for treatment with GM-CSF.

In embodiments, the present disclosure relates to a method for treating Parkinson's disease wherein biomarkers from one or more pathways including neuroinflammation signaling pathway, IL-8 signaling pathway, nitric oxide and reactive oxygen species pathways, Integrin-Linked Kinase (ILK) signaling pathway, Sirtuin signaling pathway, and/or the oxidative phosphorylation pathway are used to predict or determine the need for treatment with GM-CSF.

In embodiments, the present disclosure relates to a method for selecting a patient with Parkinson's disease and/or assessing the therapeutic response to an agent for Parkinson's disease wherein one or more of, e.g. about 1, or about 2, or about 3, or about 4, or about 5, or about 10, or about 15, or about 20, or about 25, or about 30, or about 35, or about 40, or about 45, of HMOX1, TLR2, TLR8, RELA, IKBGG, ATG3, ATG7, LRRK2, GABARAPL2, RCOR1, GGA3, ALDH1A1, RFC1, BTF3L4, WBP2, EEA1, NCBP2, PEA15, MCM5, CLTA, VPS41, SRSF4, H2AFX, CD9, RFLNB, GLB1, KRT10, ACAA1, PCK2, ATP5F1D, ATP5PB, ATP5PF, ATP5PO, COX5B, COX7C, NDUFA2, NDUFB1, NDUFB4, NDUFS2, NDUFS3, NDUFS6, NDUFS7, SDHA ATG3, ATG7, and/or GABARAPL2, optionally one or more of, e.g. about 1, or about 2, or about 3, or about 4, or about 5 of HMOX1, TLR2, TLR8, RELA, ATG7, LRRK2, and GABARAPL2, are used as a biomarker for predicting or determining the need for treatment with GM-CSF.

In embodiments, the present disclosure relates to a method for selecting a patient with Parkinson's disease and/or assessing the therapeutic response to an agent for Parkinson's disease by assaying biomarkers from one or more pathways including neuroinflammation signaling pathway, IL-8 signaling pathway, nitric oxide and reactive oxygen species pathways, Integrin-Linked Kinase (ILK) signaling pathway, Sirtuin signaling pathway, and/or the oxidative phosphorylation pathway are used to predict or determine the need for treatment with GM-CSF.

In embodiments, the biomarkers of the present disclosure are used in combination with the biomarkers described in US 2014/0349877 (the entire contents of which are incorporated by reference herein) and US 2019/0117735 (the entire contents of which are incorporated by reference herein).

In embodiments, the presence, absence or amount of one or more of the predictive clinical biomarkers is determined by detection of protein and/or nucleic acids in a biological sample of the patient.

In embodiments, the presence, absence or amount of one or more of the predictive clinical biomarkers is determined by ELISA, immunohistochemical staining, western blotting, in-cell western, immunofluorescent staining, or fluorescent activating cell sorting (FACS), or the like, in a biological sample of the patient.

In embodiments, the presence, absence, or amount of the one or more biomarkers is determined by one or more of polymerase chain reaction (PCR) amplification reaction, reverse-transcriptase PCR analysis, quantitative real-time PCR, single-strand conformation polymorphism analysis (SSCP), mismatch cleavage detection, heteroduplex analysis, deoxyribonucleic (DNA) acid sequencing, ribonucleic acid (RNA) sequencing, Northern blot analysis, in situ hybridization, array analysis, and restriction fragment length polymorphism analysis.

In embodiments, the presence, absence, or amount of the one or more biomarkers is determined by next generation sequencing (NGS) methods In embodiments, the presence, absence, or amount of the one or more biomarkers is determined by deep sequencing methods.

In embodiments, the presence, absence, or amount of the one or more biomarkers is determined by comprises ribonucleic acid (RNA) sequencing.

In embodiments, the method for determining the presence, absence or amount of one or more of the predictive clinical biomarkers is a method of characterizing a patient or selecting a patient for the treatment comprising GM-CSF.

In embodiments, the method of determining the levels of one or more of the predictive clinical biomarkers, involves assaying the biomarkers in a biological sample from the patient.

In embodiments, the present methods, e.g., the method of determining the presence, absence, levels or activity of one or more of the predictive clinical biomarkers for the purposes of patient selection, employs a biological sample, the sample selected from blood, skin sample or tissue sample, plasma, serum, pus, urine, perspiration, tears, mucus, sputum, saliva, cerebrospinal fluid (CSF) and/or other body fluids.

In embodiments, the method of patient selection is undertaken using a biological sample of the patient, where the sample is selected from blood, skin sample or tissue sample, tissue biopsy, a formalin-fixed or paraffin-embedded tissue specimen, cytological sample, cultured cells, plasma, serum, pus, urine, perspiration, tears, mucus, sputum, saliva, cerebrospinal fluid (CSF) and/or other body fluids.

In embodiments, the present methods direct patient treatment decisions. For instance, in embodiments, the method comprises the step of monitoring the expression and/or activity of one or more of the predictive clinical biomarkers during the course of treatment. In embodiments, the methods may detect a change in expression and/or activity of one or more of the predictive clinical biomarkers, and this is correlative with the disease state or therapeutic efficacy in the patient with Parkinson's disease. In such embodiments, without limitation, this directs treatment of the patient with GM-CSF agents.

In embodiments the biomarkers of the present disclosure are proteomic-based biomarkers. In other embodiments the biomarkers include one or more proteins. In another embodiment the biomarkers include one or more signaling and/or regulatory pathways within a cell. In embodiments, the expression and/or activity of the biomarkers may change (increase or decrease) with treatment. Examples of protein biomarkers include but are not limited to HMOX1, TLR2, TLR8, RELA (also referred to as RELA), IKBGG, ATG3, ATG7, LRRK2, GABARAPL2, RCOR1, GGA3, ALDH1A1, RFC1, BTF3L4, WBP2, EEA1, NCBP2, PEA15, MCM5, CLTA, VPS41, SRSF4, H2AFX, CD9, RFLNB, GLB1, KRT10, ACAA1, PCK2, ATP5F1D, ATP5PB, ATP5PF, ATP5PO, COX5B, COX7C, NDUFA2, NDUFB1, NDUFB4, NDUFS2, NDUFS3, NDUFS6, NDUFS7, SDHA, ATG3, ATG7, and/or GABARAPL2.

In embodiments, HMOX1, TLR2, TLR8, RELA, LRRK2, IKBGG, and ATP5F1D, ATP5PB, ATP5PF, ATP5PO, COX5B, COX7C, NDUFA2, NDUFB1, NDUFB4, NDUFS2, NDUFS3, NDUFS6, NDUFS7, and SDHA are downregulated during or after treatment. In embodiments, HMOX1, TLR2, TLR8, RELA, LRRK2, IKBGG, and ATP5F1D, ATP5PB, ATP5PF, ATP5PO, COX5B, COX7C, NDUFA2, NDUFB1, NDUFB4, NDUFS2, NDUFS3, NDUFS6, NDUFS7, and/or SDHA are downregulated after one month, two months, three months, four months, five months and/or after six months of treatment with GM-CSF.

In embodiments, ATG3, ATG7, and GABARAPL2 are upregulated during or after treatment with GM-CSF. In embodiments, ATG7 and GABARAPL2 are upregulated during or after treatment with GM-CSF. In embodiments, ATG3, ATG7, and GABARAPL2 are upregulated after one month, two months, three months, four months, five months and/or after six months of treatment with GM-CSF. In embodiments, ATG7 and GABARAPL2 are upregulated after one month, two months, three months, four months, five months and/or after six months of treatment with GM-CSF.

In embodiments, the neuroinflammation signaling pathway, IL-8 signaling pathway, production of nitric oxide and reactive oxygen species pathways, Integrin-Linked Kinase (ILK) signaling pathway, and the oxidative phosphorylation pathway are downregulated or inhibited during or after treatment with GM-CSF. In embodiments, the neuroinflammation signaling pathway, IL-8 signaling pathway, production of nitric oxide and reactive oxygen species pathways, Integrin-Linked Kinase (ILK) signaling pathway, and the oxidative phosphorylation pathway are downregulated and/or inhibited after one month, two months, three months, four months, five months and/or after six months of treatment with GM-CSF. In embodiments, the Sirtuin signaling pathway is upregulated and/or activated during or after treatment with GM-CSF. In embodiments, the Sirtuin signaling pathway is upregulated and/or activated after one month, two months, three months, four months, five months and/or after six months of treatment with GM-CSF.

In embodiments, the GM-CSF agents, as described herein, potentiate treatment with therapy to treat Parkinson's disease. In embodiments, GM-CSF agents, as described herein, are used to modulate the patient's immune system, e.g. by decreasing or increasing expression and/or activity of one or more of, e.g. about 1, or about 2, or about 3, or about 4, or about 5, or about 10, or about 15, or about 20, or about 25, of HMOX1, TLR2, TLR8, RELA, LRRK2, IKBGG, ATP5F1D, ATP5PB, ATP5PF, ATP5PO, COX5B, COX7C, NDUFA2, NDUFB1, NDUFB4, NDUFS2, NDUFS3, NDUFS6, NDUFS7, SDHA, ATG3, ATG7, and/or GABARAPL2. In embodiments, GM-CSF agents, as described herein, are used to modulate the patient's immune system, e.g. by decreasing or increasing expression and/or activity of various signaling pathways including the neuroinflammation signaling pathway, IL-8 signaling pathway, production of nitric oxide and reactive oxygen species pathways, Integrin-Linked Kinase (ILK) signaling pathway, and the oxidative phosphorylation pathway.

### Methods of Treatment and Disease Monitoring

In one aspect, the present disclosure relates to a method for treating Parkinson's disease comprising: administering an effective amount of a composition GM-CSF to a patient in need thereof.

In aspects, the present disclosure relates to a method for treating one or more of Parkinson's Disease, Alzheimer's disease, traumatic brain injury, stroke, amyotrophic lateral sclerosis, traumatic brain injury, progressive supranuclear palsy, down syndrome cognition, and encephalitis, comprising: administering an effective amount of a composition comprising GM-CSF to a patient in need thereof, wherein the patient is characterized by a change in expression and/or activity of various biomarkers.

In aspects, the present disclosure relates to a method for treating one or more diseases or disorders characterized by an imbalance of balance between Teff and Treg, comprising: administering an effective amount of a composition comprising GM-CSF to a patient in need thereof, wherein the patient is characterized by a change in expression and/or activity of various biomarkers.

In another aspect, the present disclosure relates to a method for treating Parkinson's disease, comprising: identifying a patient having symptoms of Parkinson's disease and determining the presence, absence or amount of any of the biomarkers of disclosed herein and administering an effective amount of a GM-CSF agent to a patient demonstrating a change in expression and/or activity of the one or more biomarkers relative to a pre-treated and/or undiseased state.

In another aspect, the present disclosure relates to methods of monitoring the regression, progression, disappearance or recurrence of symptoms of Parkinson's disease in a patient following treatment with a GM-CSF agent, the method comprising: determining the baseline expression and/or activity level of one or more of the biomarkers in a biological sample from the patient and determining the expression and/or activity level of one or more of the biomarkers in a biological sample from the patient during treatment with GM-CSF and determining if the expression and/or activity level of one or more of the biomarkers changes compared to the baseline expression and/or activity level following initiation of treatment with GM-CSF.

In aspects, the present disclosure relates to methods for treating Parkinson's disease, comprising: (a) identifying a patient undergoing or having undergone treatment with a neurological agent for neurological symptoms and presenting as failed, intolerant, resistant, or refractory to the treatment with the neurological agent; and (b) determining the presence, absence or amount of one or more of, e.g. about 1, or about 2, or about 3, or about 4, or about 5, or about 10, or about 15, or about 20, of HMOX1, TLR2, TLR8, RELA, LRRK2, IKBGG, and ATP5F1D, ATP5PB, ATP5PF, ATP5PO, COX5B, COX7C, NDUFA2, NDUFB1, NDUFB4, NDUFS2, NDUFS3, NDUFS6, NDUFS7, and SDHA, ATG3, ATG7, and/or GABARAPL2; and (c) administering an effective amount of a GM-CSF agent to a patient (i) demonstrating an increased or high expression and/or activity of one or more of, e.g. about 1, or about 2, or about 3, or about 4, or about 5, or about 10, or about 15, or about 20, of HMOX1, TLR2, TLR8, RELA, LRRK2, IKBGG, and ATP5F1D, ATP5PB, ATP5PF, ATP5PO, COX5B, COX7C, NDUFA2, NDUFB1, NDUFB4, NDUFS2, NDUFS3, NDUFS6, NDUFS7, and/or SDHA relative to a pre-treated and/or undiseased state; and/or (ii) demonstrating an decreased or low expression and/or activity of ATG3, ATG7, and/or GABARAPL2 relative to a pre-treated and/or undiseased state.

In aspects, the present disclosure relates to a method for treating Parkinson's disease in a patient, comprising: (a) identifying the patient undergoing or having undergone treatment with a neurological agent for neurological symptoms and presenting as failed, intolerant, resistant, or refractory to the treatment with the neurological agent; (b) determining an increase or decrease in expression and/or activity in biomarker(s) from one or more pathways including the neuroinflammation signaling pathway, IL-8 signaling pathway, production of nitric oxide and reactive oxygen species pathways, Integrin-Linked Kinase (ILK) signaling pathway, Sirtuin signaling pathway, and the oxidative phosphorylation pathway; and (c) administering an effective amount of a GM-CSF agent to the patient demonstrating an increased or high expression and/or activity in the neuroinflammation signaling pathway, IL-8 signaling pathway, production of nitric oxide and reactive oxygen species pathways, Integrin-Linked Kinase (ILK) signaling pathway, and the oxidative phosphorylation pathway relative to a pre-treated and/or undiseased state.

In embodiments, the present disclosure relates to a method for treating Parkinson's disease comprising: administering an effective amount of a composition comprising GM-CSF alone or in conjunction with a neurological agent and/or additional therapeutic agent to a patient in need thereof, wherein the patient is characterized by as a partial responder or a non-responder to a neurological treatment.

In embodiments, the method of treatment causes a decrease in the expression and/or activity of one or more of, e.g. about 1, or about 2, or about 3, or about 4, or about 5, or about 10, or about 15, or about 20, of HMOX1, TLR2, TLR8, RELA, LRRK2, IKBGG, and ATP5F1D, ATP5PB, ATP5PF, ATP5PO, COX5B, COX7C, NDUFA2, NDUFB1, NDUFB4, NDUFS2, NDUFS3, NDUFS6, NDUFS7, and/or SDHA.

In embodiments, the method of treatment causes an increase in the expression and/or activity of ATG3, ATG7, and/or GABARAPL2. In embodiments, the method of treatment causes an increase in the expression and/or activity of ATG7 and/or GABARAPL2.

In embodiments, the method of treatment causes a decrease in the expression and/or activity in biomarker(s) from the neuroinflammation signaling pathway, IL-8 signaling pathway, production of nitric oxide and reactive oxygen species pathways, Integrin-Linked Kinase (ILK) signaling pathway, and the oxidative phosphorylation pathway.

In embodiments, the method for monitoring the regression, progression, disappearance or recurrence of symptoms of Parkinson's disease in a patient following treatment with a GM-CSF agent, comprising: (a) determining the baseline expression and/or activity level of one or more of the biomarkers in a biological sample from the patient; (b) determining the expression and/or activity level of one or more of the biomarkers in a biological sample from the patient during treatment with GM-CSF; and (c) determining if the expression and/or activity level of one or more of the biomarkers changes compared to the baseline expression and/or activity level following initiation of treatment with GM-CSF.

In embodiments, the method of treatment prevents, treats, and/or mitigates progression and/or development of Parkinson's disease in the patient. In embodiments, the method of treatment improves the symptoms of Parkinson's disease in the patient. In embodiments, the method of treatment elicits a disease-modifying response in the patient. In other embodiments, the method of treatment elicits temporarily or permanently slows down cognitive decline in the patient. In still other embodiments, the method of treatment causes an amelioration of neurodegenerative disease symptoms. In yet other embodiments, the method of treatment slows the onset and/or development of Parkinson's disease.

In embodiments, the method of treatment decreases or mitigates reverses or prevents chronic inflammation in the central nervous system (CNS). In embodiments, the method of treatment decreases or mitigates the dysfunction of endogenous or exogenous CNS immune cells. In embodiments, the method decreases or mitigates the activation of CNS astrocytes and mononuclear phagocytes, for example perivascular macrophages and/or microglial cells.

In embodiments, the method of treatment decreases or mitigates or reverses astrogliopathy. In embodiments, the method of treatment modulates the expression and/or activity of one or more cytokines and/or proteins.

In embodiments, the method of treatment modulates or maintains or supports the glutamine-glutamate balance in the CNS. In embodiments, the method of treatment decreases or mitigates or reverses chronic microglial cell activation. In embodiments, the method of treatment decreases or reverses axonal damage.

In embodiments the method of treatment decreases or prevents protein pathologies. In embodiments, the method of treatment causes a decrease or prevents taupathy.

In embodiments, the method of treatment causes a decrease in the sequelae of Parkinson's disease in the patient relative to before treatment.

In embodiments, the patient is afflicted with a chronic, progressive disorder of the nervous system.

In embodiments, the patient is characterized by having oxidative stress, loss of neurite integrity, apoptosis, neuronal loss or/and inflammation response, cognitive impairment, cognitive decline, behavioral and personality changes, tremors, bradykinesia, rigidity, impaired posture and balance, loss of automatic movements, decrease in motor coordination, changes in speech, photophobia, difficulty controlling eye muscles, slowed saccadic eye movements, dysphagia, blepharospasm, fainting or lightheadedness due to orthostatic hypotension, dizziness, bladder control problems, well-formed visual hallucinations and delusions, changes in memory, concentration and judgement, memory loss, depression, irritability, anxiety, rapid eye movement (REM) sleep disorder, epileptic seizures, dysesthesia, numbness or tingling, spasticity, difficulty chewing or swallowing, muscle twitching and weakness in a limb, and/or prickling or tingling in feet or hands.

In embodiments, the method further comprises administering one or more additional therapeutic agents, selected from dopamine precursors such as levodopa, carbidopa (LODOSYN), dopamine agonists such as selegiline (ZELAPAR), MAO B inhibitors such as selegiline (ZELAPAR), catechol o-methyltransferase (COMT) inhibitors such as entacapone (COMTAN), anticholinergics such as benztropine (COGENTIN), amantadine, adenosine receptor antagonists (A2A receptor antagonists) such as istradefylline (NOURIANZ), and/or pimavanserin (NUPLAZID).

### Pharmaceutically Acceptable Salts and Excipients

The compositions described herein can possess a sufficiently basic functional group, which can react with an inorganic or organic acid, or a carboxyl group, which can react with an inorganic or organic base, to form a pharmaceutically acceptable salt. A pharmaceutically acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety.

Pharmaceutically acceptable salts include, by way of non-limiting example, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, pamoate, phenylacetate, trifluoroacetate, acrylate, chlorobenzoate, dinitrobenzoate, hydroxybenzoate, methoxybenzoate, methylbenzoate, o-acetoxybenzoate, naphthalene-2-benzoate, isobutyrate, phenylbutyrate, α-hydroxybutyrate, butyne-1,4-dicarboxylate, hexyne-1,4-dicarboxylate, caprate, caprylate, cinnamate, glycollate, heptanoate, hippurate, malate, hydroxymaleate, malonate, mandelate, mesylate, nicotinate, phthalate, teraphthalate, propiolate, propionate, phenylpropionate, sebacate, suberate, p-bromobenzenesulfonate, chlorobenzenesulfonate, ethylsulfonate, 2-hydroxyethylsulfonate, methylsulfonate, naphthalene-1-sulfonate, naphthalene-2-sulfonate, naphthalene-1,5-sulfonate, xylenesulfonate, and tartarate salts.

The term “pharmaceutically acceptable salt” also refers to a salt of the compositions of the present disclosure having an acidic functional group, such as a carboxylic acid functional group, and a base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or tri-alkylamines, dicyclohexylamine; tributyl amine; pyridine; N-methyl, N-ethylamine; diethylamine; triethylamine; mono-, bis-, or tris-(2-OH-lower alkylamines), such as mono-; bis-, or tris-(2-hydroxyethyl)amine, 2-hydroxy-tert-butylamine, or tris-(hydroxymethyl)methylamine, N,N-di-lower alkyl-N-(hydroxyl-lower alkyl)-amines, such as N,N-dimethyl-N-(2-hydroxyethyl)amine or tri-(2-hydroxyethyl)amine; N-methyl-D-glucamine; and amino acids such as arginine, lysine, and the like.

In embodiments, the compositions described herein are in the form of a pharmaceutically acceptable salt.

### Pharmaceutical Compositions and Formulations

In embodiments, the present disclosure pertains to pharmaceutical compositions comprising the compositions, e.g. GM-CSF and/or an additional therapeutic agent, e.g. the therapeutic agent described herein, and a pharmaceutically acceptable carrier or excipient.

In embodiments, the additional therapeutic agent comprises and/or is selected from dopamine precursors such as Levodopa, cholinesterase inhibitors such as donepezil (ARICEPT), rivastigmine (EXELON), Galantamine (RAZADYNE), atypical antipsychotics/second generation antipsychotics including serotonin-dopamin antagonists (SDAs), multi-acting receptor-targeted antipsychotics (MARTAs), and D₂ partial agonists (e.g. ABILIFY/Aripiprazol), NMDA receptor antagonist memantine, riluzole (RILUTEK), NSAIDs (non-steroidal anti-inflammatory drugs), caffein A2A receptor antagonists and CERE-120 (adeno-associated virus serotype 2-neurturin), deep brain stimulation, TNF-a antagonists including etanercept, adalimumab, infliximab, IFN-g inhibitors, TGF-b modulators, IL-33 inhibitors, IL-18 inhibitors, VEGF inhibitors, IL-1 inhibitors, inhibitors of pathological beta-amyloid (Ab) plaques, such as Ab-directed monoclonal antibodies such as aducanumab (ADUHELM), NSAIDs such as metacetamol and aspirin, anti-diabetic drugs such as linagliptin, suppressors of tau-activation such as liraglutide, miRNA's that target Ab-plaque formation and tau protein phosphorylation, α-secretase enhancers such as Ginkgo biloba and salvia miltiorrhiza, β-secretase inhibitors such as huanglian and yuanzhi, and pharmaceutically acceptable salts, acids or derivatives of any of the above.

Any pharmaceutical compositions described herein can be administered to a patient as a component of a composition that comprises a pharmaceutically acceptable carrier or vehicle. Such compositions can optionally comprise a suitable amount of a pharmaceutically acceptable excipient so as to provide the form for proper administration.

In embodiments, pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In embodiments, the pharmaceutically acceptable excipients are sterile when administered to a patient. Water is a useful excipient when any agent described herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent described herein, if desired, can also comprise minor amounts of wetting or emulsifying agents, or pH buffering agents. Other examples of suitable pharmaceutical excipients are described in Remington's Pharmaceutical Sciences 1447-1676 (Alfonso R. Gennaro eds., 19th ed. 1995), incorporated herein by reference.

The present disclosure includes the described pharmaceutical compositions (and/or additional therapeutic agents) in various formulations. Any pharmaceutical composition (and/or additional therapeutic agents) described herein can take the form of solutions, suspensions, emulsion, drops, tablets, pills, pellets, capsules, capsules containing liquids, gelatin capsules, powders, sustained-release formulations, suppositories, emulsions, aerosols, sprays, suspensions, lyophilized powder, frozen suspension, desiccated powder, or any other form suitable for use. In embodiments, the composition is in the form of a capsule. In embodiments, the composition is in the form of a tablet. In yet another embodiment, the pharmaceutical composition is formulated in the form of a soft-gel capsule. In embodiments, the pharmaceutical composition is formulated in the form of a gelatin capsule. In yet another embodiment, the pharmaceutical composition is formulated as a liquid.

Where necessary, the present pharmaceutical compositions (and/or additional therapeutic agents) can also include a solubilizing agent. Also, the agents can be delivered with a suitable vehicle or delivery device as known in the art. Combination therapies outlined herein can be co-delivered in a single delivery vehicle or delivery device.

The formulations comprising the present pharmaceutical compositions (and/or additional therapeutic agents) of the present disclosure may conveniently be presented in unit dosage forms and may be prepared by any of the methods well known in the art of pharmacy. Such methods generally include the step of bringing the therapeutic agents into association with a carrier, which constitutes one or more accessory ingredients. Typically, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

In embodiments, any pharmaceutical compositions (and/or additional therapeutic agents) described herein is formulated in accordance with routine procedures as a composition adapted for a mode of administration described herein.

Routes of administration include, for example: topical, oral, intradermal, transdermal, subcutaneous, intramuscular, intraperitoneal, intravenous, intranasal, epidural, sublingual, intranasal, intracerebral, intravaginal, rectal, or by inhalation. Administration can be local or systemic. In embodiments, the administering is by an intravenous route. The mode of administration can be left to the discretion of the practitioner, and depends in-part upon the site of the medical condition. In most instances, administration results in the release of any agent described herein onto or into the affected site.

In embodiments, the GM-CSF (and/or additional therapeutic agents) is administered via an intravenous route.

In embodiments, the pharmaceutical compositions (and/or additional therapeutic agents) described herein are formulated in accordance with routine procedures as a composition adapted for administration. For intravenous administration, suitable carriers include physiological saline, bacteriostatic water, Cremophor ELTM (BASF, Parsippany, NJ) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof.

Dosage forms suitable for parenteral administration (e.g. intravenous, intramuscular, intraperitoneal, subcutaneous and intra-articular injection and infusion) include, for example, solutions, suspensions, dispersions, emulsions, and the like. They may also be manufactured in the form of sterile solid compositions (e.g. lyophilized composition), which can be dissolved or suspended in sterile injectable medium immediately before use. They may contain, for example, suspending or dispersing agents known in the art. Formulation components suitable for parenteral administration include a sterile diluent such as water for injection, saline solution, fixed oils, polyethylene glycols, glycerin, propylene glycol or other synthetic solvents; antibacterial agents such as benzyl alcohol or methyl paraben; antioxidants such as ascorbic acid or sodium bisulfite; chelating agents such as EDTA; buffers such as acetates, citrates or phosphates; and agents for the adjustment of tonicity such as sodium chloride or dextrose.

Compositions for oral delivery can be in the form of tablets, lozenges, aqueous or oily suspensions, granules, powders, emulsions, capsules, syrups, or elixirs, for example. Orally administered compositions can comprise one or more agents, for example, sweetening agents such as fructose, aspartame or saccharin; flavoring agents such as peppermint, oil of wintergreen, or cherry; coloring agents; and preserving agents, to provide a pharmaceutically palatable preparation.

Compositions for topical delivery can be in the form of a cream, gel, ointment, lotion, spray, aqueous or oily suspensions, powders, or emulsions, for example. Increased skin permeability and penetration may be achieved by non-invasive methods, for example, with the use of any nanocarriers combined with any pharmaceutical composition (and/or additional therapeutic agents) described herein. The skin can act as a reservoir, and can be used to deliver the compositions (and/or additional therapeutic agents) described herein for more extended periods in a sustained manner.

Any pharmaceutical compositions (and/or additional therapeutic agents) described herein can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropyl cellulose, hydropropylmethyl cellulose, polyvinylpyrrolidone, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the agents described herein. The disclosure thus provides single unit dosage forms suitable for oral administration such as, but not limited to, tablets, capsules, gelcaps, and caplets that are adapted for controlled- or sustained-release.

Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In another embodiment, a controlled-release system can be placed in proximity of the target area to be treated, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

Pharmaceutical formulations preferably are sterile. Sterilization can be accomplished, for example, by filtration through sterile filtration membranes. Where the composition is lyophilized, filter sterilization can be conducted prior to or following lyophilization and reconstitution.

### Administration and Dosage

It will be appreciated that the actual dose of the composition to be administered according to the present disclosure will vary according to the particular dosage form, and the mode of administration. Many factors that may modify the action of the composition (e.g., body weight, gender, diet, time of administration, route of administration, rate of excretion, condition of the patient, drug combinations, genetic disposition and reaction sensitivities) can be taken into account by those skilled in the art. Administration can be carried out continuously or in one or more discrete doses within the maximum tolerated dose. Optimal administration rates for a given set of conditions can be ascertained by those skilled in the art using conventional dosage administration tests.

In embodiments, the GM-CSF is administered at a total dose of about 125 μg, about 150 μg, or about 200 μg, or about 250 μg, or about 300 μg, or about 350 μg. In embodiments, the GM-CSF is administered at a total dose of about 250 μg.

In embodiments, the GM-CSF is administered at a dose of about 125 μg, about 150 μg, or about 200 μg, or about 250 μg, or about 300 μg, or about 350 μg.

In embodiments, the GM-CSF is administered twice daily.

In embodiments, the GM-CSF is sargramostim, administered at a dose of about 125 μg, twice daily.

### Combination Therapy and Additional Therapeutic Agents

In embodiments, the pharmaceutical composition of the present disclosure is co-administered in conjunction with additional agent(s), for example a neurological agent and/or additional therapeutic agent. Co-administration can be simultaneous or sequential.

In embodiments, the additional neurological agent and/or additional therapeutic agent and the GM-CSF of the present disclosure are administered to a patient simultaneously. The term “simultaneously” as used herein, means that the neurological agent and/or additional therapeutic agent and the GM-CSF are administered with a time separation of no more than about 60 minutes, such as no more than about 30 minutes, no more than about 20 minutes, no more than about 10 minutes, no more than about 5 minutes, or no more than about 1 minute. Administration of the neurological agent and/or additional therapeutic agent and the GM-CSF can be by simultaneous administration of a single formulation (e.g., a formulation comprising the additional therapeutic agent and the GM-CSF composition) or of separate formulations (e.g., a first formulation including the neurological agent and/or additional therapeutic agent and a second formulation including the GM-CSF composition).

Co-administration does not require the therapeutic agents to be administered simultaneously, if the timing of their administration is such that the pharmacological activities of the neurological agent and/or additional therapeutic agent and the GM-CSF overlap in time, thereby exerting a combined therapeutic effect. For example, the neurological agent and/or additional therapeutic agent and the targeting moiety, the GM-CSF composition can be administered sequentially. The term “sequentially” as used herein means that the neurological agent and/or additional therapeutic agent and the GM-CSF are administered with a time separation of more than about 60 minutes. For example, the time between the sequential administration of the neurological agent and/or additional therapeutic agent and the GM-CSF can be more than about 60 minutes, more than about 2 hours, more than about 5 hours, more than about 10 hours, more than about 1 day, more than about 2 days, more than about 3 days, more than about 1 week apart, more than about 2 weeks apart, or more than about one month apart. The optimal administration times will depend on the rates of metabolism, excretion, and/or the pharmacodynamic activity of the additional therapeutic agent and the GM-CSF being administered. Either the neurological agent and/or additional therapeutic agent or the GM-CSF composition may be administered first.

Co-administration also does not require the therapeutic agents to be administered to the patient by the same route of administration. Rather, each therapeutic agent can be administered by any appropriate route, for example, parenterally or non-parenterally.

In embodiments, the GM-CSF described herein acts synergistically when co-administered with the neurological agent and/or additional therapeutic agent. In such embodiments, the GM-CSF composition and the neurological agent and/or additional therapeutic agent may be administered at doses that are lower than the doses employed when the agents are used in the context of monotherapy.

### Biological Samples

In embodiments, the biological sample is selected from a biopsy, a tissue and/or a body fluid.

In embodiments, the biological sample is selected from blood, skin sample or tissue sample, tissue biopsy, a formalin-fixed or paraffin-embedded tissue specimen, cytological sample, cultured cells, plasma, serum, pus, urine, perspiration, tears, mucus, sputum, saliva and/or other body fluids.

In embodiments, the biological sample is selected from blood, skin sample or tissue sample, plasma, serum, pus, urine, perspiration, tears, mucus, sputum, saliva, cerebrospinal fluid (CSF) and/or other body fluids.

In embodiments, the biological sample is/or comprises monocytes. In embodiments, the biological sample is/or comprises monocyte population

In embodiments, the biological sample is a peripheral blood lymphocyte (PBL), e.g. isolated by leukapheresis and centrifugal elutriation.

In embodiments, the biological sample is a lymphocyte population, e.g. isolated by leukapheresis and centrifugal elutriation.

### Kits

The disclosure also provides kits that can detect the presence or absence a biomarker described herein.

A typical kit of the invention comprises various reagents including, for example, an agent to detect a biomarker described herein. In embodiments, the kit comprises one or more of reagents for detection, including those useful in various detection methods, described herein. In embodiments, the kit comprises materials necessary for the evaluation, including, e.g., welled plates, syringes, and the like. In embodiments, the kit further comprises a label or printed instructions instructing the use of described reagents.

In embodiments, there is provided kits for measuring the present biomarkers in the biological sample. In embodiments, the kit includes a multi-well sample plate that is coated with immobilized capture antibodies that bind to the biomarkers; detection antibodies covalently linked to an enzyme wherein the detection antibodies also bind to the biomarkers; a colored or fluorescent product that is catalyzed by the enzyme attached to the detection antibody; and appropriate buffers. In embodiments, the kit has an ELISA plate which is specific for detecting one of the biomarkers. In embodiments, the kit detects one or more of, e.g. about 1, or about 2, or about 3, or about 4, or about 5, of LRRK2, HMOX1, TLR2, TLR8, RELA, ATG7, and GABARAPL2.

In embodiments, there is provided a companion diagnostic, complementary diagnostic, or co-diagnostic test kit, comprising: (a) an array of nucleic acids or proteins suitable for detection of one or more of e.g. about 1, or about 2, or about 3, or about 4, or about 5, or about 10, or about 15, or about 20 of HMOX1, TLR2, TLR8, RELA, LRRK2, IKBGG, and ATP5F1D, ATP5PB, ATP5PF, ATP5PO, COX5B, COX7C, NDUFA2, NDUFB1, NDUFB4, NDUFS2, NDUFS3, NDUFS6, NDUFS7, and SDHA, ATG3, ATG7, and GABARAPL2; and (b) instructions for use.

In embodiments, there is provided a companion diagnostic, complementary diagnostic, or co-diagnostic test kit, comprising reagents and instructions for use in one or more of methods described herein.

Sequences

SEQ ID NO: 1 is the amino acid sequence of wild type GM-CSF:
APARSPSPSTQPWEHVNAIQEARRLLNLSRDTAAEMNETVEVISEMFDL
QEPTCLQTRLELYKQGLRGSLTKLKGPLTMMASHYKQHCPPTPETSCATQIITFESFKE
NLKDFLLVIPFDCWEPVQE.
SEQ ID NO: 2 is the amino acid sequence of sargramostim:
APARSPSPSTQPWEHVNAIQEALRLLNLSRDTAAEMNETVEVISEMFDL
QEPTCLQTRLELYKQGLRGSLTKLKGPLTMMASHYKQHCPPTPETSCATQIITFESFKE
NLKDFLLVIPFDCWEPVQE.
SEQ ID NO: 3 is the amino acid sequence of molgramostim:
APARSPSPSTQPWEHVNAIQEARRLLNLSRDTAAEMNETVEVISEMFDL
QEPTCLQTRLELYKQGLRGSLTKLKGPLTMMASHYKQHCPPTPETSCATQIITFESFKE
NLKDFLLVIPFDCWEPVQE
SEQ ID NO: 4 is the amino acid sequence of regramostim:
APARSPSPSTQPWEHVNAIQEARRLLNLSRDTAAEMNETVEVISEMFDL
QEPTCLQTRLELYKQGLRGSLTKLKGPLTMMASHYKQHCPPTPETSCATQTTFESFKE
NLKDFLLVIPFDCWEPVQE

### Definitions

The following definitions are used in connection with the invention disclosed herein. Unless defined otherwise, all technical and scientific terms used herein have the same meaning as commonly understood to one of skill in the art to which this invention belongs.

An “effective amount,” when used in connection with an agent effective for the treatment of a coronavirus infection is an amount that is effective for treating or mitigating a coronavirus infection.

As used herein, “a,” “an,” or “the” can mean one or more than one. Further, the term “about” when used in connection with a referenced numeric indication means the referenced numeric indication plus or minus up to 10% of that referenced numeric indication. For example, the language “about 50” covers the range of 45 to 55.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

### EXAMPLES

### Example 1: Monocyte Proteomic Profile after the Sargramostim Treatment

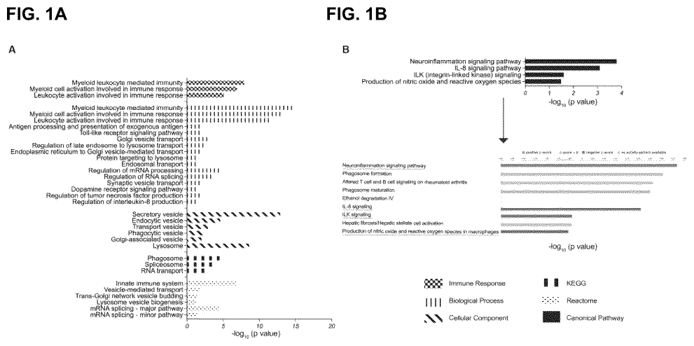

To obtain a detailed understanding of changes in the monocyte proteomic profile after the sargramostim treatment, functional and pathway enrichment analyses of differentially regulated proteins, at 2 and 6 months, after treatment were performed. At 2 months, multiple immune processes were enriched upon sargramostim treatment, including myeloid leukocyte mediated immunity (p=1.33E-08), myeloid cell activation involved in immune response (p=1.30E-07), and leukocyte activation involved in immune response (p=7.25E-06) (FIG. 1A). Similarly, Kyoto Encyclopedia of Genes and Genomes (KEGG) and Reactome analyses showed phagosome (p=2.74E-04) and innate immune system (p=1.29E-07) enrichments, respectively (FIG. 1A). In addition, there was an enrichment of inflammation processes including regulation of interleukin-8 (IL-8) (p=3.64E-02) and tumor necrosis factor (TNF) production pathways (p=1.73E-02) (FIG. 1A). Further, Ingenuity Pathway Analysis (IPA) showed inhibition of neuroinflammation signaling (p=1.43E-04), IL-8 (p=8.8E-04), integrin-linked kinase (ILK) (p=2.8E-02), nitric oxide and reactive oxygen species (ROS) (p=3.35E-02) pathways (FIG. 1A). Additionally, sargramostim affected endosomal (p=3.14E-02), Golgi vesicle (p=1.60E-03), endoplasmic reticulum to Golgi vesicle-mediated (p=1.02E-02), protein targeting to lysosome (p=2.78E-02), and regulation of late endosome to lysosome (p=3.88E-03) transport mechanisms (FIG. 1A). Further, cellular component and Reactome analyses showed enrichment of secretory vesicle (p=1.14E-13), endocytic vesicle (p=2.37E-05), transport vesicle (p=1.18E-03), phagocytic vesicle (p=5.16E-03), Golgi-associated vesicle (p=9.24E-03), lysosome (p=2.59E-09), and trans-Golgi network vesicle budding (p=4.19E-02) (FIG. 1A). Sargramostim treatment also induced changes in biological processes related to the RNA processing such as regulation of mRNA processing (p=2.16E-05) and regulation of RNA splicing (p=3.96E-04) (FIG. 1A). Similarly, KEGG and Reactome tests showed pathways affecting RNA processing (including RNA transport, p=3.70E-02), mRNA splicing (major and minor pathways, p=3.54E-05 and p=4.31E-02, respectively), and spliceosome (1.24E-03) (FIG. 1A).

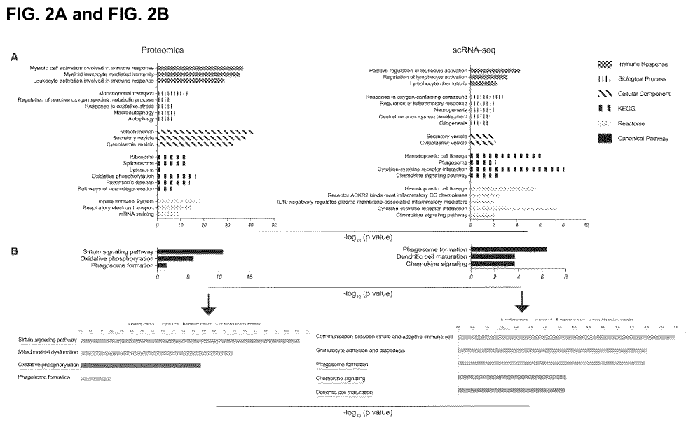

Proteomic and scRNA-seq analyses at 6 months of sargramostim treatment also showed enrichment of immune processes, including monocyte-macrophage mediated immunity (p=6.03E-34), activation (p=2.29E-35), and innate immune responses (p=2.91E-27). Moreover, regulation of leukocyte activation (p=5.05E-03), lymphocyte chemotaxis (p=5.02E-03), and regulation of lymphocyte activation (p=6.540E-04) were shown (FIG. 2A). Similarly, proteomic Reactome analysis showed enrichment of innate immunity (p=3.66E-19) and phagosome formation (p=3.28E-02) (FIG. 2A and FIG. 2B). IPA of 6 month-scRNA-seq data sets showed chemokine signaling (p=1.84E-04), dendritic cell maturation (p=2.03E-04), and phagosome formation (p=3.73E-07) enrichments (FIG. 2A and FIG. 2B). In addition, there was an enrichment of regulation of ROS metabolism (p=3.22E-03), oxidative stress (p=7.07E-04), and oxygen containing compounds (p=1.16E-03) (FIG. 2A). These data indicated antioxidant effects that followed sargramostim treatment. Interestingly, IPA of 6 month-proteomic data showed inhibition of oxidative phosphorylation (p=1.36E-06). This suggested that control of ROS production can occur after sargramostim treatment (FIG. 2B). Moreover, 6 month-proteomic data showed an enrichment of mitochondria function (p=8.26E-40) as the organelle responsible for respiratory electron transport pathway (p=5.29E-15) enriched in Reactome analysis (FIG. 2A). Additionally, cellular component analysis of the both proteomic and scRNA-seq data sets showed enrichment of secretory (p=1.59E-38) and cytoplasmic (p=6.56E-03) vesicles (FIG. 2A). Six month-proteomic data sets demonstrated enrichment of autophagy (p=4.63E-04) and macroautophagy (1.51E-05) (biological process analysis) and activation of the sirtuin signaling pathway (IPA, p=2.19E-11) (FIG. 2A and FIG. 2B). The 6 month-scRNA-seq data also showed enrichment of pathways linked to regulation of inflammatory responses (p=5.87E-03), IL-10 negatively regulates plasma membrane-associated inflammatory mediators (p=9.67E-03), and receptor ACKR2 binds most inflammatory CC chemokines (p=3.16E-03) (FIG. 2A).

### Example 2: Expression of Genes and Proteins Following Sargramostim Treatment

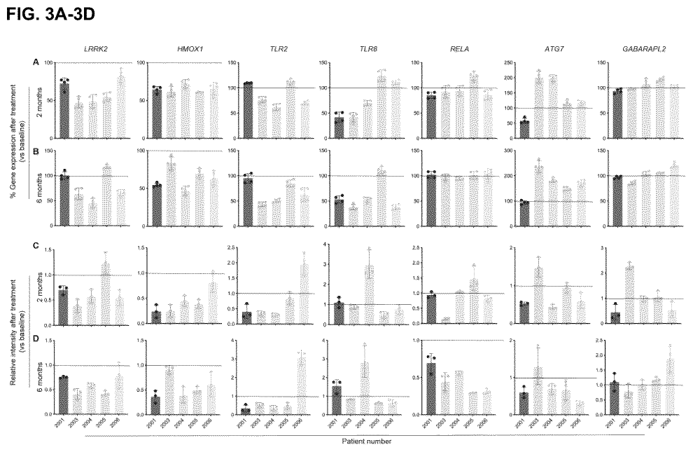

ddPCR and Western blotting were used to assess the expression of genes and proteins as biomarkers for predicting disease progression and therapeutic response following sargramostim treatment. The individual subject's baseline and treatment protein expression of LRRK2, HMOX1, TLR2, TLR8, RELA, ATG7, and GABARAPL2 at 2 and 6 months were compared after starting the sargramostim treatment. At 2 months, 4/5 of patients displayed significant decreased protein expression of LRRK2 and HMOX1, and 3/5 of patients displayed significant decreased expression of TLR2 protein following sargramostim initiation (FIG. 3A). In addition, 3/5 of patients showed decreased protein expression of TLR8 and NF-κB with significant downregulation in 1/3 of patients; subject 2005 for TLR8 and subject 2003 for NF-κB (FIG. 3A). Additionally, 1 patient displayed increased expression of ATG7 protein (subject 2003) and the same subject displayed significant increased expression of GABARAPL2 protein (FIG. 3A). At 6 months, 4/5 patients showed significant downregulation of LRRK2 and TLR2 (FIG. 3B). Notably, 5/5 of patients showed decreased protein expression of HMOX1. The downregulation was significant in 3/5 of patients (FIG. 3B). Similarly, 5/5 of patients showed decreased expression of NF-κB and the downregulation was significant in 4/5 of patients (FIG. 3B). In addition, downregulation of TLR8 protein was observed in 2/5 of patients (FIG. 3B). Only subject 2003 displayed limited increased expression of ATG7 protein while 3/5 of patients showed an upregulation of GABARAPL2 reaching near significance in 2/3 patients (subjects 2005 and 2006) (FIG. 3B). At the genetic level, the individual patient's baseline and treatment gene expression of LRRK2, HMOX1, TLR2, TLR8, RELA, ATG7, and GABARAPL2 at 2 and 6 months after initiating sargramostim were compared. At 2 months, 5/5 patients displayed significant decreased gene expression of LRRK2 and HMOX1, and 3/5 patients showed significant decreased expression of TLR2 and TLR8 genes after sargramostim treatment (FIG. 3C). Despite 4/5 patients having displayed significant decreased gene expression of NF-κB, the downregulation did not exceed 15% compared to the baseline (FIG. 3C). Interestingly, 4/5 patients showed significant upregulation of ATG7 gene, but the upregulation did not exceed 17%, compared to the baseline, in 2/4 subjects (subjects 2005 and 2006) (FIG. 3C). Similarly, 3/5 patients displayed significant upregulation of GABARAPL2 gene, but the upregulation did not exceed 17% compared to the baseline in the 3 subjects (FIG. 3C). At 6 months, 5/5 patients showed significant downregulation of HMOX1 and TLR2 genes (FIG. 3D). 3/5 and 4/5 patients showed significant downregulation of LRRK2 and TLR8 genes, respectively (FIG. 3D). Only 2/5 patients displayed significant decreased genetic expression of NF-κB with downregulation not exceeding 5% compared to the baseline (FIG. 3D). Interestingly, 4/5 patients showed significant upregulation of ATG7 gene (FIG. 3D). In accordance with 2 month-ddPCR data, 3/5 patients displayed significant increased gene expression of GABARAPL2, but the upregulation did not exceed 6%, compared to the baseline, in 2/3 subjects (subjects 2004 and 2005) (FIG. 3D).

### Example 3: Integrated scRNA-Seq and Proteomic Data after Sargramostim Treatment

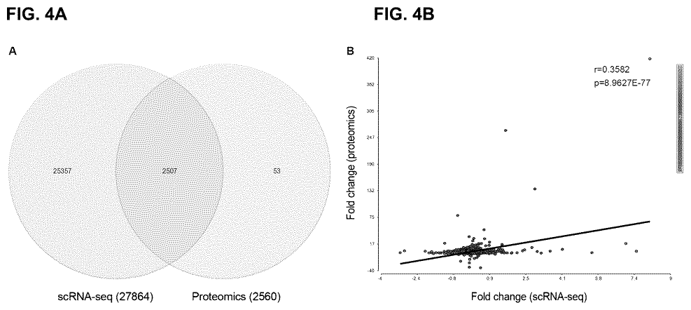

The overlapping genes between scRNA-seq and proteomic datasets of subjects 2003, 2004, and 2005 are illustrated using Venn diagram which show the number of genes identified in each dataset and the number of overlapped genes in both datasets (FIG. 4A). Pearson product-moment correlation coefficient between scRNA-seq and proteomic overlapping genes (r=0.3582, p=8.9627E-77) showed significant moderate positive association between the overlapping genes in both datasets (FIG. 4B). This indicates that the expression of multiple genes in both datasets has been changed in the same direction (downregulation or upregulation).

### Example 4: Western Blot and ddPCR Correlations with Clinical MDS-UPDRS III Scores

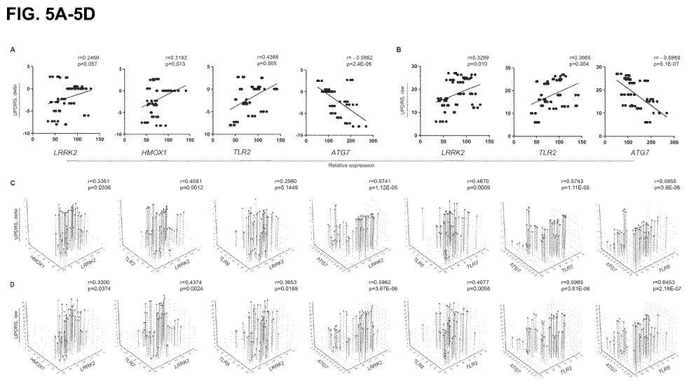

Correlating the expression of potential biomarkers (at the gene or protein level) with MDS-UPDRS III scores of PD patients could be used to identify the potentiality of using clinical biomarkers to predict the disease progression and therapeutic response after immune modulator therapies such as sargramostim. Therefore, correlation analysis of the raw and change in MDS-UPDRS III score with the protein levels of selected biomarkers was performed. Positive correlations were shown between change in MDS-UPDRS III score and protein level of LRRK2 (r=0.3534, p=0.017), HMOX1 (r=0.0881, p=0.565), TLR8 (r=0.1531, p=0.315), and NF-κB (r=0.4258, p=0.004) (FIG. 5A), suggesting that motor function is improved with decreased expression of any of these proteins. In addition, negative correlations were shown between change in MDS-UPDRS III score and protein level of ATG7 (r=−0.2440, p=0.106) and GABARAPL2 (r=−0.1376, p=0.367) (FIG. 5A), Similarly, positive correlations were shown between raw MDS-UPDRS III score and protein level of LRRK2 (r=0.2659, p=0.077), HMOX1 (r=0.1109, p=0.468), TLR2 (r=0.3704, p=0.012), and NF-κB (r=0.1847, p=0.224) (FIG. 5B), while negative correlations were shown between raw MDS-UPDRS III score and protein level of ATG7 (r=−0.3002, p=0.045) and GABARAPL2 (r=−0.1110, p=0.468) (FIG. 5B). LRRK2 and HMOX1 were both able to predict a change in MDS-UPDRS III score with variation in the change of MDS-UPDRS III score at 12.5% (FIG. 5C). However, in this model LRRK2 provided a stronger effect (β=0.3594, p=0.0222) than HMOX1. The effect of LRRK2 and NF-κB was significant (p=0.0139) with 18.4% of change in MDS-UPDRS III values (FIG. 5C). The effects of NF-κB and GABARAPL2 showed significant effect on change in MDS-UPDRS III score (p=0.0128) with 18.7% effect on the change of MDS-UPDRS III score (FIG. 5C). The effects of TLR2 and TLR8 showed no significant effect on the change in MDS-UPDRS III score (p=0.567) (FIG. 5C), nor did TLR8 and ATG7 have an effect on the change in MDS-UPDRS III score (p=0.2403) (FIG. 5C). Further analysis for the effects on raw MDS-UPDRS III score showed significant effects by LRRK2/HMOX1, LRRK2/NF-κB, and NF-κB/GABARAPL2 (p=0.2093, 0.2123, and 0.4761, respectively) (FIG. 5D), whereas the other 2 pairs of predictors (TLR2/TLR8 and TLR8/ATG7) showed significant effects on raw MDS-UPDRS III score (p=0.0314 and p=0.0046, respectively) (FIG. 5D).

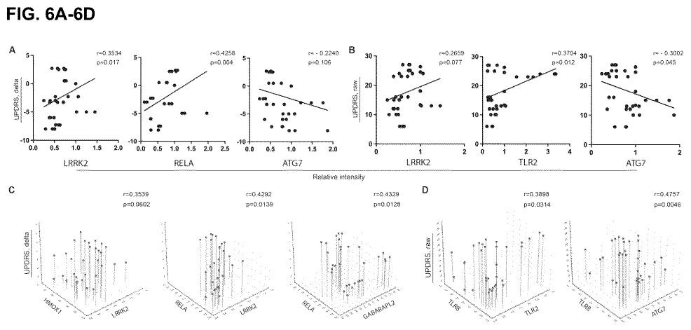

Similarly, correlations were made between the MDS-UPDRS III scores and gene expression of selected biomarkers. This included LRRK2 (r=0.2469, p=0.057), HMOX1 (r=0.3193, p=0.013), TLR2 (r=0.4388, p=0.000), and TLR8 (r=0.0385, p=0.770) positive correlations with the change in MDS-UPDRS-III scores (FIG. 6A). Each demonstrated relationships between decreased gene expression and improved motor function. Negative correlations were made between the change in MDS-UPDRS III scores and ATG7 (r=−0.5662, p=0.000) and GABARAPL2 (r=−0.0360, p=0.785) (FIG. 6A). Each demonstrated relationships between increased gene expression and improved motor function. Similarly, replicate correlations were seen between raw MDS-UPDRS III scores and LRRK2 (r=0.3299, p=0.010), HMOX1 (r=0.1598, p=0.223), and TLR2 (r=0.3665, p=0.004) (FIG. 6B). Negative correlations were affirmed between raw MDS-UPDRS III scores and ATG7 (r=−0.5960, p=0.000) and GABARAPL2 (r=−0.0115, p=0.930) (FIG. 6B). The data proved significant (p=0.0336) with variation in MDS-UPDRS III score change at 11.2% with both predictors (FIG. 6C). HMOX1 was a stronger predictor (β=0.2611, p=0.0747) although it failed to reach significance. Each unit increase in ddPCR HMOX1 expression increased the change in MDS-UPDRS III score by 0.0409 point. The effect of LRRK2 and TLR2 was significant (p=0.0012) with 21% of change in MDS-UPDRS III values (FIG. 6C). TLR2 provided a greater effect (β=0.4013, p=0.0018) than did LRRK2. The model predicted that 1 unit increase in ddPCR TLR2 expression increased the change in MDS-UPDRS III score by 0.0503 point. On the other hand, LRRK2 and TLR8 showed no significant effect on the change in MDS-UPDRS III scores (p=0.1449) (FIG. 6C). However, LRRK2 and ATG7 were linked to the change in MDS-UPDRS III score (p=1.12E-05) with 33% effect on the score change (FIG. 6C). ATG7 provided a greater effect (β=−0.6326, p=0.0000) than LRRK2 with an increase in 1 unit of ddPCR relative expression in ATG7 to affect the MDS-UPDRS III score change by 0.0400 point. The effect of TLR2 and TLR8 was significant (p=0.0009) with a 21.8% change in the MDS-UPDRS III score (FIG. 6C). TLR2 provided more significant effect (β=0.5124, p=0.0002) over TLR8. The model predicts that 1 unit increase in the ddPCR relative expression of TLR2 would increase the change in MDS-UPDRS III score by 0.0643 point. TLR2 and ATG7 were significantly linked to the MDS-UPDRS III score change (p=1.11E-05) with a 33% score effect (FIG. 6C). ATG7 more than TLR2 provided the greatest influence on the MDS-UPDRS III score change (β=−0.4850, p=0.0012); where an increase in 1 unit of ddPCR in ATG7 decreased the MDS-UPDRS III score change by 0.0307 point. TLR8 and ATG7 also showed significant effect on the change in MDS-UPDRS III score (p=3.8E-06) with a 35.5% effect (FIG. 6C).

### EQUIVALENTS

Those skilled in the art will recognize, or be able to ascertain, using no more than routine experimentation, numerous equivalents to the embodiments described specifically herein. Such equivalents are intended to be encompassed in the scope of the following claims.

### INCORPORATION BY REFERENCE

All patents and publications referenced herein are hereby incorporated by reference in their entireties.

As used herein, all headings are simply for organization and are not intended to limit the disclosure in any manner. The content of any individual section may be equally applicable to all sections.

What is claimed is:

1. A method of selecting a patient for treatment with an agent for Parkinson's disease comprising:
determining the presence, absence or amount of one or more biomarkers in a biological sample from the patient, wherein:
the patient is suitable for the treatment if demonstrating a change in the expression and/or activity of the biomarkers relative to a pre-treated and/or undiseased state, and
the agent comprises an effective amount of a granulocyte-macrophage colony-stimulating factor (GM-CSF).

2. The method of claim 1, wherein the biomarker is selected from HMOX1, TLR2, TLR8, RELA, IKBGG, ATG3, ATG7, Leucine-rich repeat serine/threonine protein kinase 2 (LRRK2), GABARAPL2, RCOR1, GGA3, ALDH1A1, RFC1, BTF3L4, WBP2, EEA1, NCBP2, PEA15, MCM5, CLTA, VPS41, SRSF4, H2AFX, CD9, RFLNB, GLB1, KRT10, ACAA1, PCK2, ATP5F1D, ATP5PB, ATP5PF, ATP5PO, COX5B, COX7C, NDUFA2, NDUFB1, NDUFB4, NDUFS2, NDUFS3, NDUFS6, NDUFS7, SDHA, ATG3, ATG7, and GABARAPL2, optionally wherein the biomarker is selected from HMOX1, TLR2, TLR8, RELA, ATG7, LRRK2, and GABARAPL2.

3. The method of claim 1, wherein one or more of HMOX1, TLR2, TLR8, RELA, LRRK2, IKBGG, and ATP5F1D, ATP5PB, ATP5PF, ATP5PO, COX5B, COX7C, NDUFA2, NDUFB1, NDUFB4, NDUFS2, NDUFS3, NDUFS6, NDUFS7, and SDHA are downregulated during or after treatment with GM-CSF, optionally wherein one or more of HMOX1, TLR2, TLR8, RELA, and LRRK2 are downregulated during or after treatment with GM-CSF.

4. The method of claim 3, wherein one or more of HMOX1, TLR2, TLR8, RELA, LRRK2, IKBGG, and ATP5F1D, ATP5PB, ATP5PF, ATP5PO, COX5B, COX7C, NDUFA2, NDUFB1, NDUFB4, NDUFS2, NDUFS3, NDUFS6, NDUFS7, and SDHA are downregulated after one month, two months, three months, four months, five months and/or after six months of treatment with GM-CSF, optionally wherein one or more of HMOX1, TLR2, TLR8, RELA, and LRRK2 are downregulated after one month, two months, three months, four months, five months and/or after six months of treatment with GM-CSF.

5. The method of claim 1, wherein one or more of ATG3, ATG7, and GABARAPL2 are upregulated during or after treatment with GM-CSF, optionally wherein one or more of ATG7 and GABARAPL2 are upregulated during or after treatment with GM-CSF.

6. The method of claim 5, wherein all of ATG3, ATG7 and GABARAPL2 are upregulated during or after treatment with GM-CSF, optionally wherein both of ATG7 and GABARAPL2 are upregulated during or after treatment with GM-CSF.

7. The method of claim 5 or 6, wherein one or more of ATG3, ATG7, and GABARAPL2 are upregulated after one month, two months, three months, four months, five months and/or after six months of treatment with GM-CSF, optionally wherein ATG7 and GABARAPL2 are upregulated after one month, two months, three months, four months, five months and/or after six months of treatment with GM-CSF.

8. The method of claim 1, wherein:
(i) one or more of HMOX1, TLR2, TLR8, RELA, LRRK2, IKBGG, and ATP5F1D, ATP5PB, ATP5PF, ATP5PO, COX5B, COX7C, NDUFA2, NDUFB1, NDUFB4, NDUFS2, NDUFS3, NDUFS6, NDUFS7, and SDHA are downregulated during or after treatment with GM-CSF, optionally wherein one or more of HMOX1, TLR2, TLR8, RELA, and LRRK2 are downregulated during or after treatment with GM-CSF, and
(ii) one or more of ATG3, ATG7 and GABARAPL2 are upregulated during or after treatment with GM-CSF, optionally wherein one or more of ATG7 and GABARAPL2 are upregulated during or after treatment with GM-CSF.

9. The method of any one of claims 1-8, wherein the biomarker is associated with one or more pathways selected from a neuroinflammation signaling pathway, IL-8 signaling pathway, production of nitric oxide and reactive oxygen species pathways, Integrin-Linked Kinase (ILK) signaling pathway, Sirtuin signaling pathway, and oxidative phosphorylation pathway.

10. The method of claim 9, wherein the biomarker associated with a neuroinflammation signaling pathway, IL-8 signaling pathway, production of nitric oxide and reactive oxygen species pathways, Integrin-Linked Kinase (ILK) signaling pathway, and the oxidative phosphorylation pathway are downregulated or inhibited during or after treatment with GM-CSF.

11. The method of claim 10, wherein the biomarker associated with a neuroinflammation signaling pathway, IL-8 signaling pathway, production of nitric oxide and reactive oxygen species pathways, Integrin-Linked Kinase (ILK) signaling pathway, and the oxidative phosphorylation pathway are downregulated or inhibited after one month, two months, three months, four months, five months and/or after six months of treatment with GM-CSF.

12. A method for treating Parkinson's disease in a patient, the method comprising the steps of:
(a) identifying the patient having symptoms of Parkinson's disease; and
(b) determining the presence, absence or amount of one or more biomarkers in a biological sample from the patient; and
(c) administering an effective amount of GM-CSF agent to the patient demonstrating a change in expression and/or activity of the one or more biomarkers relative to a pre-treated and/or undiseased state.

13. The method of claim 12, wherein the biomarker is selected from HMOX1, TLR2, TLR8, RELA, IKBGG, ATG3, ATG7, LRRK2, RCOR1, GGA3, ALDH1A1, RFC1, BTF3L4, WBP2, EEA1, NCBP2, PEA15, MCM5, CLTA, VPS41, SRSF4, H2AFX, CD9, RFLNB, GLB1, KRT10, ACAA1, PCK2, ATP5F1D, ATP5PB, ATP5PF, ATP5PO, COX5B, COX7C, NDUFA2, NDUFB1, NDUFB4, NDUFS2, NDUFS3, NDUFS6, NDUFS7, SDHA, ATG3, ATG7, and GABARAPL2, optionally wherein the biomarker is selected from HMOX1, TLR2, TLR8, RELA, ATG7, LRRK2, and GABARAPL2.

14. The method of claim 12, wherein one or more of HMOX1, TLR2, TLR8, RELA, LRRK2, IKBGG, and ATP5F1D, ATP5PB, ATP5PF, ATP5PO, COX5B, COX7C, NDUFA2, NDUFB1, NDUFB4, NDUFS2, NDUFS3, NDUFS6, NDUFS7, and SDHA are downregulated during or after treatment with GM-CSF, optionally wherein one or more of HMOX1, TLR2, TLR8, RELA, ATG7, LRRK2, and GABARAPL2 are downregulated during or after treatment with GM-CSF.

15. The method of claim 14, wherein one or more of HMOX1, TLR2, TLR8, RELA, LRRK2, IKBGG, and ATP5F1D, ATP5PB, ATP5PF, ATP5PO, COX5B, COX7C, NDUFA2, NDUFB1, NDUFB4, NDUFS2, NDUFS3, NDUFS6, NDUFS7, and SDHA are downregulated after one month, two months, three months, four months, five months and/or after six months of treatment with GM-CSF, optionally wherein one or more of HMOX1, TLR2, TLR8, RELA, and LRRK2 are downregulated after one month, two months, three months, four months, five months and/or after six months of treatment with GM-CSF.

16. The method of claim 12, wherein one or more of ATG3, ATG7, and GABARAPL2 are upregulated during or after treatment with GM-CSF, optionally wherein one or more of ATG7 and GABARAPL2 are upregulated during or after treatment with GM-CSF.

17. The method of claim 16, wherein all of ATG3, ATG7, and GABARAPL2 are upregulated during or after treatment with GM-CSF, optionally wherein both of ATG7 and GABARAPL2 are upregulated during or after treatment with GM-CSF.

18. The method of claim 16 or 17, wherein ATG3, ATG7, and GABARAPL2 are upregulated after one month, two months, three months, four months, five months and/or after six months of treatment with GM-CSF, optionally wherein ATG7 and GABARAPL2 are upregulated after one month, two months, three months, four months, five months and/or after six months of treatment with GM-CSF.

19. The method of claim 12, wherein:
(i) one or more of HMOX1, TLR2, TLR8, RELA, LRRK2, IKBGG, and ATP5F1D, ATP5PB, ATP5PF, ATP5PO, COX5B, COX7C, NDUFA2, NDUFB1, NDUFB4, NDUFS2, NDUFS3, NDUFS6, NDUFS7, and SDHA are downregulated during or after treatment with GM-CSF, optionally wherein one or more of HMOX1, TLR2, TLR8, RELA, and LRRK2 are downregulated during or after treatment with GM-CSF, and
(ii) one or more of ATG3, ATG7, and GABARAPL2 are upregulated during or after treatment with GM-CSF, optionally wherein one or more of ATG7 and GABARAPL2 are upregulated during or after treatment with GM-CSF.

20. The method of any one of claims 12-19, wherein the biomarker is associated with one or more pathways selected from a neuroinflammation signaling pathway, IL-8 signaling pathway, production of nitric oxide and reactive oxygen species pathways, Integrin-Linked Kinase (ILK) signaling pathway, Sirtuin signaling pathway, and oxidative phosphorylation pathway.

21. The method of claim 20, wherein the biomarker associated with a neuroinflammation signaling pathway, IL-8 signaling pathway, production of nitric oxide and reactive oxygen species pathways, Integrin-Linked Kinase (ILK) signaling pathway, and the oxidative phosphorylation pathway are downregulated or inhibited during or after treatment with GM-CSF.

22. The method of claim 21, wherein the biomarker associated with a neuroinflammation signaling pathway, IL-8 signaling pathway, production of nitric oxide and reactive oxygen species pathways, Integrin-Linked Kinase (ILK) signaling pathway, and the oxidative phosphorylation pathway are downregulated or inhibited after one month, two months, three months, four months, five months and/or after six months of treatment with GM-CSF.

23. A method for treating Parkinson's disease in a patient, the method comprising the steps of:
(a) identifying the patient undergoing or having undergone treatment with a neurological agent for neurological symptoms and presenting as failed, intolerant, resistant, or refractory to the treatment with the neurological agent; and
(b) determining the presence, absence or amount of one or more of HMOX1, TLR2, TLR8, RELA, LRRK2, IKBGG, and ATP5F1D, ATP5PB, ATP5PF, ATP5PO, COX5B, COX7C, NDUFA2, NDUFB1, NDUFB4, NDUFS2, NDUFS3, NDUFS6, NDUFS7, and SDHA, ATG3, ATG7, and GABARAPL2, optionally one or more of HMOX1, TLR2, TLR8, RELA, LRRK2, ATG7, and GABARAPL2; and
(c) administering an effective amount of a GM-CSF agent to the patient
(i) demonstrating a decreased or low expression and/or activity of one or more of HMOX1, TLR2, TLR8, RELA, LRRK2, IKBGG, and ATP5F1D, ATP5PB, ATP5PF, ATP5PO, COX5B, COX7C, NDUFA2, NDUFB1, NDUFB4, NDUFS2, NDUFS3, NDUFS6, NDUFS7, and/or SDHA, optionally one or more of HMOX1, TLR2, TLR8, RELA, and LRRK2, relative to a pre-treated and/or undiseased state; and/or
(ii) demonstrating an increased or high expression and/or activity of one or more of ATG3, ATG7, and/or GABARAPL2, optionally ATG7 and/or GABARAPL2, relative to a pre-treated and/or undiseased state.

24. A method for treating Parkinson's disease in a patient, the method comprising the steps of:
(a) identifying the patient undergoing or having undergone treatment with an neurological agent for neurological symptoms and presenting as failed, intolerant, resistant, or refractory to the treatment with the neurological agent;
(b) determining a decrease in expression and/or activity in biomarker from one or more pathways including the neuroinflammation signaling pathway, IL-8 signaling pathway, production of nitric oxide and reactive oxygen species pathways, Integrin-Linked Kinase (ILK) signaling pathway, and the oxidative phosphorylation pathway and/or an increase in expression and/or activity in biomarker from Sirtuin signaling pathway; and
(c) administering an effective amount of a GM-CSF agent to the patient demonstrating a decreased or low expression and/or activity in the neuroinflammation signaling pathway, IL-8 signaling pathway, production of nitric oxide and reactive oxygen species pathways, Integrin-Linked Kinase (ILK) signaling pathway, and the oxidative phosphorylation pathway relative to a pre-treated and/or undiseased state.

25. A method for monitoring the regression, progression, disappearance or recurrence of symptoms of Parkinson's disease in a patient following treatment with a GM-CSF agent, the method comprising the steps of:
(a) determining a baseline expression and/or activity level of one or more of the biomarkers at a first time point in a biological sample from the patient;
(b) determining the expression and/or activity level of one or more of the biomarkers at a second and subsequent time point in a biological sample; and
(c) determining if the expression and/or activity level of one or more of the biomarkers changes between the first and second time points,
wherein the one or more biomarkers are selected from HMOX1, TLR2, TLR8, RELA, LRRK2, IKBGG, and ATP5F1D, ATP5PB, ATP5PF, ATP5PO, COX5B, COX7C, NDUFA2, NDUFB1, NDUFB4, NDUFS2, NDUFS3, NDUFS6, NDUFS7, and SDHA, ATG3, ATG7, and GABARAPL2, optionally wherein the one or more biomarkers are selected from HMOX1, TLR2, TLR8, RELA, LRRK2, ATG7, and GABARAPL2.

26. The method of any one of claims 1-25, further comprising administering an effective amount of a drug or therapeutic to treat Parkinson's disease.

27. The method of any one of claims 1-26, wherein the patient is characterized by having one or more of oxidative stress, loss of neurite integrity, apoptosis, neuronal loss or/and inflammation response, cognitive impairment, cognitive decline, behavioral and personality changes, tremors, bradykinesia, rigidity, impaired posture and balance, loss of automatic movements, decrease in motor coordination, changes in speech, photophobia, difficulty controlling eye muscles, slowed saccadic eye movements, dysphagia, blepharospasm, fainting or lightheadedness due to orthostatic hypotension, dizziness, bladder control problems, well-formed visual hallucinations and delusions, changes in memory, concentration and judgement, memory loss, depression, irritability, anxiety, rapid eye movement (REM) sleep disorder, epileptic seizures, dysesthesia, numbness or tingling, spasticity, difficulty chewing or swallowing, muscle twitching and weakness in a limb, and/or prickling or tingling in feet or hands.

28. The method of any one of claims 1-27, wherein the presence, absence, or amount of the one or more biomarkers is determined by detection of protein and/or nucleic acids.

29. The method of any one of claims 1-28, wherein the presence, absence, or amount of the one or more biomarkers is determined by one or more of ELISA, Luminex multiplex assay, immunohistochemical staining, western blotting, in-cell western, immunofluorescent staining, or fluorescent activating cell sorting (FACS).

30. The method of any one of claims 1-29, wherein the presence, absence, or amount of the one or more biomarkers is determined by one or more of droplet-digital PCR (ddPCR), reverse-transcriptase PCR analysis, quantitative real-time PCR, single-strand conformation polymorphism analysis (SSCP), mismatch cleavage detection, heteroduplex analysis, deoxyribonucleic (DNA) acid sequencing, ribonucleic acid (RNA) sequencing, Northern blot analysis, in situ hybridization, array analysis, and restriction fragment length polymorphism analysis.

31. The method of any one of claims 1-30, wherein the presence, absence, or amount of the one or more biomarkers is determined by single-cell RNA sequencing and/or next generation sequencing (NGS) methods.

32. The method of claim 30, wherein the presence, absence, or amount of the one or more biomarkers is determined by ribonucleic acid (RNA) sequencing, optionally single-cell RNA sequencing.

33. The method of any one of claims 1-32, wherein the biological sample is/or comprises blood, skin sample or tissue sample, plasma, serum, pus, urine, perspiration, tears, mucus, sputum, saliva, cerebrospinal fluid (CSF) and/or other body fluids.

34. The method of any one of claims 1-32, wherein the biological sample is/or comprises monocytes.

35. The method of any one of claims 1-32, wherein the biological sample is/or comprises monocyte population.

36. The method of any one of claims 1-35, wherein the method prevents, treats, and/or mitigates progression and/or development of Parkinson's disease in the patient.

37. The method of any one of claims 1-36, wherein the method elicits a disease-modifying response.

38. The method of any one of claims 1-37, wherein the method elicits temporarily or permanently slows down cognitive decline.

39. The method of any one of claims 1-38, wherein the method causes an amelioration of neurodegenerative disease symptoms.

40. The method of any one of claims 1-39, wherein the method slows the onset and/or development of the neurodegenerative disease or disorder.

41. The method of any one of claims 1-40, wherein the method reverses or prevents chronic inflammation in the central nervous system (CNS).

42. The method of any one of claims 1-41, wherein the method decreases or mitigates the dysfunction of endogenous or exogenous CNS immune cells.

43. The method of any one of claims 1-42, wherein the method decreases or mitigates the activation of CNS astrocytes and mononuclear phagocytes.

44. The method of any one of claims 1-43, where the mononuclear phagocytes comprise perivascular macrophages and/or microglial cells.

45. The method of any one of claims 1-44, wherein the method decreases or mitigates or reverses astrogliopathy.

46. The method of any one of claims 1-45, wherein the method modulates or maintains or supports a glutamine-glutamate balance in the CNS.

47. The method of any one of claims 1-46, wherein the method decreases or mitigates or reverses chronic microglial cell activation.

48. The method of any one of claims 1-47, wherein the method decreases or reverses axonal damage.

49. The method of any one of claims 1-48, wherein the method decreases or prevents excessive production and/or signaling of one or more inflammatory cytokines and/or proteins.

50. The method of any one of claims 1-49, wherein the method decreases or prevents the formation of protein plaques.

51. The method of any one of claims 1-50, wherein the method causes a decrease or prevents taupathy.

52. The method of any one of claims 1-51, wherein the GM-CSF has an amino acid sequence of SEQ ID NO: 1, or a variant of at least about 90%, or at least about 93%, or at least about 95%, or at least about 97%, or at least about 98% identity thereto.

53. The method of any one of claims 1-51, wherein the GM-CSF has an amino acid sequence of one of SEQ ID NO: 2, SEQ ID NO: 3, and SEQ ID NO: 4, or a variant of at least about 90%, or at least about 93%, or at least about 95%, or at least about 97%, or at least about 98% identity thereto.

54. The method of any one of claims 52-53, wherein the GM-CSF is one of molgramostim, sargramostim, and regramostim.

55. The method of claim 54, wherein the GM-CSF is sargramostim.

56. The method of any one of claims 1-55, wherein the GM-CSF is administered to via a subcutaneous administration.

57. The method of any one of claims 1-56, wherein the method further comprises administering one or more additional therapeutic agents, selected from dopamine precursors such as levodopa, carbidopa (LODOSYN), dopamine agonists such as selegiline (ZELAPAR), MAO B inhibitors such as selegiline (ZELAPAR), catechol o-methyltransferase (COMT) inhibitors such as entacapone (COMTAN), anticholinergics such as benztropine (COGENTIN), amantadine, adenosine receptor antagonists (A2A receptor antagonists) such as istradefylline (NOURIANZ), and/or pimavanserin (NUPLAZID).

58. A method for treating Parkinson's disease, comprising:
(a) selecting a patient having Parkinson's disease and one or more of changed expression and/or activity of one or more biomarkers relative to an undiseased state; and
(b) administering an effective amount of a composition comprising GM-CSF to the patient,
wherein the one or more biomarkers are selected from HMOX1, TLR2, TLR8, RELA, LRRK2, IKBGG, and ATP5F1D, ATP5PB, ATP5PF, ATP5PO, COX5B, COX7C, NDUFA2, NDUFB1, NDUFB4, NDUFS2, NDUFS3, NDUFS6, NDUFS7, and SDHA, ATG3, ATG7, and GABARAPL2, optionally wherein the one or more biomarkers are selected from HMOX1, TLR2, TLR8, RELA, LRRK2, ATG7, and GABARAPL2.

59. The method of claim 58, wherein a changed expression and/or activity of the one or more biomarkers directs discontinued administration of GM-CSF.

60. The method of any one of claims 58-59, wherein the levels of any of the biomarkers are assayed in a biological sample from the patient.

61. The method of claim 60, wherein the biological sample comprises blood, tissue sample, plasma, serum, pus, urine, perspiration, tears, mucus, sputum, saliva, cerebrospinal fluid (CSF) and/or other body fluids.

62. The method of any one of claims 58-61, wherein the method prevents, treats, and/or mitigates progression and/or development of Parkinson's disease.

63. The method of any one of claims 58-62, wherein the method improves the symptoms of Parkinson's disease in the patient.

64. The method of any one of claims 58-63, wherein the method causes a decrease in the sequelae of Parkinson's disease in the patient relative to before treatment.

65. The method of any one of claims 58-64, wherein the GM-CSF has an amino acid sequence of SEQ ID NO: 1, or a variant of at least about 90%, or at least about 93%, or at least about 95%, or at least about 97%, or at least about 98% identity thereto.

66. The method of any one of claims 58-64, wherein the GM-CSF has an amino acid sequence of one of SEQ ID NO: 2, SEQ ID NO: 3, and SEQ ID NO: 4, or a variant of at least about 90%, or at least about 93%, or at least about 95%, or at least about 97%, or at least about 98% identity thereto.

67. The method of claim 65 or 66, wherein the GM-CSF is one of molgramostim, sargramostim, and regramostim.

68. The method of claim 67, wherein the GM-CSF is sargramostim.

69. The method of any one of claims 58-68, wherein the GM-CSF is administered via an intravenous route.

70. The method of any one of claims 58-69, wherein the method further comprises administering one or more additional therapeutic agents, selected from dopamine precursors such as levodopa, carbidopa (LODOSYN), dopamine agonists such as selegiline (ZELAPAR), MAO B inhibitors such as selegiline (ZELAPAR), catechol o-methyltransferase (COMT) inhibitors such as entacapone (COMTAN), anticholinergics such as benztropine (COGENTIN), amantadine, adenosine receptor antagonists (A2A receptor antagonists) such as istradefylline (NOURIANZ), and/or pimavanserin (NUPLAZID).

71. A companion diagnostic, complementary diagnostic, or co-diagnostic test kit, comprising:
(a) an array of nucleic acids or proteins suitable for detection of one or more of HMOX1, TLR2, TLR8, RELA, LRRK2, IKBGG, and ATP5F1D, ATP5PB, ATP5PF, ATP5PO, COX5B, COX7C, NDUFA2, NDUFB1, NDUFB4, NDUFS2, NDUFS3, NDUFS6, NDUFS7, and SDHA, ATG3, ATG7, and GABARAPL2, optionally one or more of HMOX1, TLR2, TLR8, RELA, LRRK2, ATG7, and GABARAPL2; and
(b) instructions for use.

72. A companion diagnostic, complementary diagnostic, or co-diagnostic test kit, comprising reagents and instructions for use in one or more of claims 1-70.
